(19) United States
(12) Patent Application Publication
(10) Pub. No.: US 2023/0117388 A1
(43) Pub. Date: Apr. 20, 2023

(54) METHODS AND MATERIALS FOR EXPANDING TUMOR INFILTRATING GAMMA-DELTA T CELLS

(71) Applicant: University of Pittsburgh - Of the Commonwealth System of Higher Education, Pittsburgh, PA (US)

(72) Inventors: Michael T. Lotze, Pittsburgh, PA (US); Pranav Murthy, Pittsburgh, PA (US)

(21) Appl. No.: 17/966,444

(22) Filed: Oct. 14, 2022

Related U.S. Application Data
(60) Provisional application No. 63/257,805, filed on Oct. 20, 2021.

(51) Int. Cl. A61K 35/17; C12N 5/0783; A61P 35/00

(52) U.S. Cl. CPC A61K 35/17; C12N 5/0636; A61P 35/00; C12N 2501/2302; C12N 2501/2304; C12N 2501/2315; A61K 2039/5158

(57) ABSTRACT

This document provides methods and materials for expanding tumor infiltrating γδ T cells (e.g., tumor infiltrating γδ T cells) in culture. For example, methods and materials for expanding large numbers of tumor infiltrating γδ T cells (e.g., tumor infiltrating γδ T cells that are predominantly Vδ1⁺) from tissue obtained from a mammal having cancer (e.g., a tumor sample), an autoimmune condition, or an infection are provided. Populations of such tumor infiltrating γδ T cells and methods and materials for using such tumor infiltrating γδ T cells and/or such populations to treat cancer within a mammal (e.g., a human) also are provided.

### CROSS-REFERENCE TO RELATED APPLICATIONS

This application claims the benefit of U.S. Provisional Application Ser. No. 63/257,805, filed Oct. 20, 2021. The disclosure of the prior application is considered part of (and is incorporated by reference in) the disclosure of this application.

### BACKGROUND

### 1. Technical Field

This document relates to methods and materials for expanding tumor infiltrating gamma-delta (γδ) T cells (e.g., tumor infiltrating γδ T cells) in culture. For example, this document provides methods and materials for expanding large numbers of tumor infiltrating γδ T cells (e.g., tumor infiltrating γδ T cells that are predominantly Vδ1⁺) from tissue obtained from a mammal having cancer (e.g., a tumor sample). This document also provides populations of such tumor infiltrating γδ T cells and methods and materials for using such tumor infiltrating γδ T cells and/or such populations to treat cancer within a mammal (e.g., a human).

### 2. Background Information

Cancer immunotherapies including adoptive cell therapy (ACT) with tumor infiltrating lymphocytes (TIL) depend on T cell effector functions. These αβ T cell receptor (TCR) expressing cells target cancer cells through recognition of peptide or lipid antigens presented by major histocompatibility complex (MHC) Class I and II and MHC-like CD1 molecules. TIL therapies that include lymphodepletion, adoptive transfer of ex vivo expanded autologous TIL, and post infusion administration of high dose interleukin-2 (IL-2) has provided durable complete responses in patients with treatment refractory metastatic melanoma, cervical cancer, and other epithelial cancers. With current TIL therapy protocols providing objective clinical response and in particular, complete responses, in many treated patients, improvements in the understanding of the mechanisms of treatment response can help broaden the application of these treatments (Dafni et al., Ann. Oncol., 30:1902-1913 (2019)).

Clinical manifestation of cancer often occurs following years of cancer immune editing with the emergence of poorly immunogenic tumor cell variants, many of which have lost Class I MHC molecules (Schreiber et al., Science, 331:1565-1570 (2011)). Despite efforts to reinvigorate immune responses with ACT, genomic instability of cancer cells promotes Darwinian selection processes associated with mutational downregulation or complete loss of immune reactive tumor associated peptide antigens that provide a means of immune escape (Dudley et al., J Clin. Oncol., 23:2346-2357 (2005); Khong et al., Nat. Immunol., 3:999-1005 (2002); Zitvogel et al., Nat. Rev. Immunol., 6:715-727 (2006); and Orlando et al., Nat. Med., 24:1504-1506 (2018)).

As noted, immune evasion is also mediated by reduced expression or lack of MHC-Class 1 antigen presentation that is pervasive across several solid tumors and limits the efficacy of αβ T cell immunotherapy (Dhatchinamoorthy et al., Front. Immunol., 12:636568 (2021); Tran et al., N. Engl. J. Med., 375:2255-2262 (2016); and Chowell et al., Science, 359:582-587 (2018)). More recently, T cell intrinsic factors, including functional exhaustion associated with lack of effective co-stimulation, inhibitory receptor expression and abrogation of stem cell like memory differentiation dictate persistence and response to immunotherapy (Ahmadzadeh et al., Blood, 114:1537-1544 (2009); Baitsch et al., J Clin. Invest., 121:2350-2360 (2011); Miller et al., Nat. Immunol., 20:326-336 (2019); Sade-Feldman et al., Cell, 175:998-1013 e1020 (2018); Jansen et al., Nature, 576:465-470 (2019); and Krishna et al., Science, 370:1328-1334 (2020)). Therapeutic interventions that can overcome challenges inherent to tumor cell immune escape and suppression paradigms can further improve immunotherapy treatment outcomes.

γδ TCR expressing cells are an evolutionarily conserved lymphocytic subset whose MHC-unrestricted recognition of pathogen derived or host cell non-peptide metabolites and stress antigens provide compelling opportunities to discern their utility in immunosurveillance and cancer immunotherapy (Vantourout et al., Nat. Rev. Immunol., 13:88-100 (2013); Silva-Santos et al., Nat. Rev. Immunol., 15:683-691 (2015); Silva-Santos et al., Nat. Rev. Cancer, 19:392-404 (2019); Sebestyen et al., Nat. Rev. Drug Discov., 19:169-184 (2020); and Ribot et al., Nat. Rev. Immunol., 21:221-232 (2021)). γδ T cells, especially Vδ1⁺ cells, are predominantly tissue resident immune effectors that display diverse roles in mediating TCR- and natural cytotoxicity receptor (NCR)-dependent tumor surveillance. As such, they coordinate and mediate both innate and adaptive immune responses (Vantourout et al., Nat. Rev. Immunol., 13:88-100 (2013); Silva-Santos et al., Nat. Rev. Immunol., 15:683-691 (2015); Silva-Santos et al., Nat. Rev. Cancer, 19:392-404 (2019); Sebestyen et al., Nat. Rev. Drug Discov., 19:169-184 (2020); Ribot et al., Nat. Rev. Immunol., 21:221-232 (2021); and Davey et al., Trends Immunol., 39:446-459 (2018)). The presence of these cells is associated with better outcomes in patients with many types of cancer. For example, patients with leukemia recovering an increased number of γδ T cells following bone marrow transplantation experienced greater long-term survival (Godder et al., Bone Marrow Transplant., 39:751-757 (2007)). Furthermore, a meta-analysis of infiltrating immune cell gene expression signatures of 25 solid tumor types from the cancer genome atlas (TCGA) identified γδ T cells to be the most significant cell type associated with favorable prognosis (Gentles et al., Nat. Med., 21:938-945 (2015)). Early and ongoing efforts targeting phosphoantigen reactive, blood resident Vγ9Vδ2 cells have established the clinical feasibility and safety of γδ cancer cell therapy (Sebestyen et al., Nat. Rev. Drug Discov., 19:169-184 (2020)).

### SUMMARY

This document provides methods and materials for expanding tumor infiltrating γδ T cells (e.g., tumor infiltrating γδ T cells) in culture. For example, this document provides methods and materials for expanding tumor infiltrating γδ T cells obtained from tissue (e.g., a tumor sample) to obtain large numbers (e.g., greater than 1×10⁷, greater than 1×10⁸, greater than 5×10⁸, or greater than 1×10⁹) of tumor infiltrating γδ T cells (e.g., tumor infiltrating γδ T cells that are predominantly Vδ1⁺) within, for example, 25 to 30 days.

As described herein, γδ T cells obtained from tumor tissue (and/or healthy tissue that is within 30 mm of a tumor) can be expanded in vitro using a combination of cytokines (e.g., IL-2 plus IL-4 plus IL-15 (IL-2/IL-4/IL-15)) to produce populations of tumor infiltrating γδ T cells having desired percentages of cells having desired phenotypes. For example, this document provides methods and materials for expanding tumor infiltrating γδ T cells by culturing a first population containing tumor infiltrating γδ T cells in the presence of IL-2 for 5 to 15 days (e.g., 6 to 15 days, 7 to 15 days, 8 to 15 days, 9 to 15 days, 9 to 13 days, 10 to 12 days, or 7 to 10 days) to produce a second population of cells, and subsequently culturing the second population of cells in the presence of IL-2, IL-4, and IL-15 (and optionally PBMCs such as irradiated allogeneic PBMCs and optionally an anti-CD3 agonistic antibody) for 8 to 21 days (e.g., 10 to 21 days, 12 to 21 days, 14 to 21 days, 8 to 18 days, 8 to 16 days, 8 to 14 days, 10 to 20 days, 10 to 18 days, 12 to 18 days, 10 to 16 days, 12 to 16 days, or 13 to 15 days) to produce an expanded population of tumor infiltrating γδ T cells. In some cases, a population of expanded tumor infiltrating γδ T cells can be obtained by (a) obtaining a tissue sample containing a tumor and/or healthy tissue that was within 30 mm of a tumor, (b) obtaining a first cell population containing tumor infiltrating γδ T cells from that tissue, (c) optionally enriching that first cell population so that the resulting enriched population contains a higher ratio of tumor infiltrating γδ T cells to total CD3⁺ cells, and (d) culturing the first cell population (or the optional enriched population) in the presence of IL-2, IL-4, IL-15, PBMCs (e.g., irradiated PBMCs), and an anti-CD3 antibody for 8 to 21 days (e.g., 10 to 21 days, 12 to 21 days, 14 to 21 days, 8 to 18 days, 8 to 16 days, 8 to 14 days, 10 to 20 days, 10 to 18 days, 12 to 18 days, 10 to 16 days, 12 to 16 days, or 13 to 15 days) to obtain a population of expanded tumor infiltrating γδ T cells.

In some cases, greater than 85 percent of the CD3⁺ cells of an expanded population provided herein can be γδ TCR⁺ cells, less than 10 percent of the CD3⁺ cells of that population can be αβ TCR⁺ cells, less than 10 percent of the CD45⁺ cells of that population can be NK cells, greater than 30 percent of the γδ TCR⁺ cells of that population can be Vδ1⁺ cells, less than 60 percent of the γδ TCR⁺ cells of that population can be Vδ1⁻Vδ2⁻ cells, less than 25 percent of the γδ TCR⁺ cells of that population can be Vδ2⁺ cells, greater than 70 percent of the γδ TCR⁺ cells of that population can be TEM cells, less than 25 percent of the γδ TCR⁺ cells of that population can be T_{EMRA} cells, as high as 10 percent of the γδ TCR⁺ cells of that population can be CD69⁺ CD103⁺ Tissue resident memory (T_{RM}) cells, as high as 50 percent of the γδ TCR⁺ cells of that population can be CD56⁺ cells, from 1 to 40 percent of the γδ TCR⁺ cells of that population can be CD137⁺ cells, less than 25 percent of the γδ TCR⁺ cells of that population can be PD-1⁺ cells, from 5 to 40 percent of the γδ TCR⁺ cells of that population can be BTLA⁺ cells, greater than 60 percent of the γδ TCR⁺ cells of that population can be NKG2D⁺ cells, and greater than 20 percent of the γδ TCR⁺ cells of that population can be NKp46⁺ cells.

As also described herein, the populations of tumor infiltrating γδ T cells provided herein can be administered to a mammal (e.g., human) having cancer to treat cancer within that mammal. For example, a population of tumor infiltrating γδ T cells provided herein can be administered (e.g., intravenously administered) to a mammal (e.g., a human) having cancer as an adoptive cellular therapy to treat that cancer either alone or in combination with (a) tumor infiltrating αβ T cells and/or (b) one or more therapeutic agents such as one or more checkpoint inhibitors (e.g., anti-PD-1 antibodies and/or anti-PD-L1 antibodies), IL-2, one or more lymphodepleting chemotherapy agents (e.g., cyclophosphamide and/or fludarabine), one or more tumor infiltrating lymphocyte enhancement agents (e.g., CpG and/or oncolytic viruses such as vaccinia viruses), brachytherapy, or combinations thereof. In such cases, the administered tumor infiltrating γδ T cells can provide effective immune responses against cancer cells within the mammal, thereby reducing the number of cancer cells within the mammal.

In general, one aspect of this document features a method for producing a cell population comprising γδ T cells. The method comprises (or consists essentially of or consists of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69⁺ CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

In another aspect, this document features an isolated cell population comprising (or consisting essentially of or consisting of) polyclonal γδ T cells, wherein the population comprises greater than 1×10⁸ γδ T cells. Greater than 85 percent of the CD3⁺ cells the cell population can be γδ TCR⁺ cells. Less than 10 percent of the CD3⁺ cells of the cell population can be αβ TCR⁺ cells. Less than 10 percent of the CD45⁺ cells of the cell population can be NK cells. Greater than 30 percent of the γδ TCR⁺ cells of the cell population can be Vδ1⁺ cells. Less than 60 percent of the γδ TCR⁺ cells of the cell population can be Vδ1⁻Vδ2⁻ cells. Less than 25 percent of the γδ TCR⁺ cells of the cell population can be Vδ2⁺ cells. Greater than 70 percent of the γδ TCR⁺ cells of the cell population can be TEM cells. Less than 25 percent of the γδ TCR⁺ cells of the cell population can be T_{EMRA} cells. Less than 10 percent of the γδ TCR⁺ cells of the cell population can be CD69⁺ CD103⁺ T_{RM} cells. From 1 to 10 percent of the γδ TCR⁺ cells of the cell population can be CD69⁺ CD103⁺ T_{RM} cells. Less than 50 percent of the γδ TCR⁺ cells of the cell population can be CD56⁺ cells. From 1 to 50 percent of the γδ TCR⁺ cells of the cell population can be CD56⁺ cells. From 1 to 40 percent of the γδ TCR⁺ cells of the cell population can be CD137⁺ cells. Less than 25 percent of the γδ TCR⁺ cells of the cell population can be PD-1⁺ cells. From 5 to 40 percent of the γδ TCR⁺ cells of the cell population can be BTLA⁺ cells. Greater than 60 percent of the γδ TCR⁺ cells of the cell population can be NKG2D⁺ cells. Greater than 20 percent of the γδ TCR⁺ cells of the cell population can be NKp46⁺ cells. The cells of the cell population can be human cells. The γδ T cells can be tumor infiltrating γδ T cells. The cell population can be a cell population that was produced using a method for producing a cell population comprising γδ T cells as described in any statement or combination of statements from the following paragraph.

The method can comprise (or can consist essentially of or can consist of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69⁺ CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

In another aspect, this document features a method for providing a mammal with γδ T cells. The method comprises (or consists essentially of or consists of) administering, to a mammal, a cell population produced as described in any statement or combination of statements from the preceding paragraph. The mammal can be a human. The mammal can be a mammal having cancer. The cells of the first cell population can be allogenic or autologous to the mammal administered the cell population. The method can comprise administering αβ T cells to the mammal.

In another aspect, this document features a method for providing a mammal with γδ T cells. The method comprises (or consists essentially of or consists of) administering a cell population (e.g., an isolated cell population) to a mammal. The mammal can be a human. The mammal can be a mammal having cancer, an autoimmune condition, or an infection. The cells of the cell population can be allogenic or autologous to the mammal. The method can comprise administering αβ T cells to the mammal. The cell population (e.g., isolated cell population) can comprise (or can consist essentially of or can consist of) polyclonal γδ T cells, wherein the population comprises greater than 1×10⁸ γδ T cells. Greater than 85 percent of the CD3⁺ cells the cell population can be γδ TCR⁺ cells. Less than 10 percent of the CD3⁺ cells of the cell population can be αβ TCR⁺ cells. Less than 10 percent of the CD45⁺ cells of the cell population can be NK cells. Greater than 30 percent of the γδ TCR⁺ cells of the cell population can be Vδ1⁺ cells. Less than 60 percent of the γδ TCR⁺ cells of the cell population can be Vδ1⁻Vδ2⁻ cells. Less than 25 percent of the γδ TCR⁺ cells of the cell population can be Vδ2⁺ cells. Greater than 70 percent of the γδ TCR⁺ cells of the cell population can be TEM cells. Less than 25 percent of the γδ TCR⁺ cells of the cell population can be T_{EMRA} cells. Less than 10 percent of the γδ TCR⁺ cells of the cell population can be CD69⁺ CD103⁺ T_{RM} cells. From 1 to 10 percent of the γδ TCR⁺ cells of the cell population can be CD69⁺ CD103⁺ T_{RM} cells. Less than 50 percent of the γδ TCR⁺ cells of the cell population can be CD56⁺ cells. From 1 to 50 percent of the γδ TCR⁺ cells of the cell population can be CD56⁺ cells. From 1 to 40 percent of the γδ TCR⁺ cells of the cell population can be CD137⁺ cells. Less than 25 percent of the γδ TCR⁺ cells of the cell population can be PD-1⁺ cells. From 5 to 40 percent of the γδ TCR⁺ cells of the cell population can be BTLA⁺ cells. Greater than 60 percent of the γδ TCR⁺ cells of the cell population can be NKG2D⁺ cells. Greater than 20 percent of the γδ TCR⁺ cells of the cell population can be NKp46⁺ cells. The cells of the cell population can be human cells. The γδ T cells can be tumor infiltrating γδ T cells. The cell population can be a cell population that was produced using a method for producing a cell population comprising γδ T cells as described in any statement or combination of statements from the following paragraph.

The method can comprise (or can consist essentially of or can consist of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69+CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

In another aspect, this document features a method for treating cancer. The method comprises (consists essentially of or consists of) administering, to a mammal having cancer, a cell population produced as described in any statement or combination of statements from the preceding paragraph. The mammal can be a human. The cells of the first cell population can be allogenic or autologous to the mammal having cancer. The method can comprise administering αβ T cells to the mammal.

In another aspect, this document features a method for treating cancer. The method comprises (consists essentially of or consists of) administering a cell population (e.g., an isolated cell population) to a mammal having cancer. The mammal can be a human. The cells of the cell population can be allogenic or autologous to the mammal having cancer. The method can comprise administering αβ T cells to the mammal. The cell population (e.g., isolated cell population) can comprise (or can consist essentially of or can consist of) polyclonal γδ T cells, wherein the population comprises greater than 1×10⁸ γδ T cells. Greater than 85 percent of the CD3⁺ cells the cell population can be γδ TCR⁺ cells. Less than 10 percent of the CD3⁺ cells of the cell population can be αβ TCR⁺ cells. Less than 10 percent of the CD45⁺ cells of the cell population can be NK cells. Greater than 30 percent of the γδ TCR⁺ cells of the cell population can be Vδ1⁺ cells. Less than 60 percent of the γδ TCR⁺ cells of the cell population can be Vδ1⁻Vδ2⁻ cells. Less than 25 percent of the γδ TCR⁺ cells of the cell population can be Vδ2⁺ cells. Greater than 70 percent of the γδ TCR⁺ cells of the cell population can be TEM cells. Less than 25 percent of the γδ TCR⁺ cells of the cell population can be T_{EMRA} cells. Less than 10 percent of the γδ TCR⁺ cells of the cell population can be CD69⁺ CD103⁺ T_{RM} cells. From 1 to 10 percent of the γδ TCR⁺ cells of the cell population can be CD69⁺ CD103⁺ T_{RM} cells. Less than 50 percent of the γδ TCR⁺ cells of the cell population can be CD56⁺ cells. From 1 to 50 percent of the γδ TCR⁺ cells of the cell population can be CD56⁺ cells. From 1 to 40 percent of the γδ TCR⁺ cells of the cell population can be CD137⁺ cells. Less than 25 percent of the γδ TCR⁺ cells of the cell population can be PD-1⁺ cells. From 5 to 40 percent of the γδ TCR⁺ cells of the cell population can be BTLA⁺ cells. Greater than 60 percent of the γδ TCR⁺ cells of the cell population can be NKG2D⁺ cells. Greater than 20 percent of the γδ TCR⁺ cells of the cell population can be NKp46⁺ cells. The cells of the cell population can be human cells. The γδ T cells can be tumor infiltrating γδ T cells. The cell population can be a cell population that was produced using a method for producing a cell population comprising γδ T cells as described in any statement or combination of statements from the following paragraph.

The method can comprise (or can consist essentially of or can consist of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69+CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

In another aspect, this document features a method for treating an autoimmune condition. The method comprises (consists essentially of or consists of) administering a cell population (e.g., an isolated cell population) to a mammal having an autoimmune condition. The mammal can be a human. The cells of the cell population can be allogenic or autologous to the mammal having the autoimmune condition. The method can comprise administering αβ T cells to the mammal. The cell population (e.g., isolated cell population) can comprise (or can consist essentially of or can consist of) polyclonal γδ T cells, wherein the population comprises greater than 1×10⁸ γδ T cells. Greater than 85 percent of the CD3⁺ cells the cell population can be γδ TCR⁺ cells. Less than 10 percent of the CD3⁺ cells of the cell population can be αβ TCR⁺ cells. Less than 10 percent of the CD45⁺ cells of the cell population can be NK cells. Greater than 30 percent of the γδ TCR⁺ cells of the cell population can be Vδ1⁺ cells. Less than 60 percent of the γδ TCR⁺ cells of the cell population can be Vδ1⁻Vδ2⁻ cells. Less than 25 percent of the γδ TCR⁺ cells of the cell population can be Vδ2⁺ cells. Greater than 70 percent of the γδ TCR⁺ cells of the cell population can be TEM cells. Less than 25 percent of the γδ TCR⁺ cells of the cell population can be T_{EMRA} cells. Less than 10 percent of the γδ TCR⁺ cells of the cell population can be CD69⁺ CD103⁺ T_{RM} cells. From 1 to 10 percent of the γδ TCR⁺ cells of the cell population can be CD69⁺ CD103⁺ T_{RM} cells. Less than 50 percent of the γδ TCR⁺ cells of the cell population can be CD56⁺ cells. From 1 to 50 percent of the γδ TCR⁺ cells of the cell population can be CD56⁺ cells. From 1 to 40 percent of the γδ TCR⁺ cells of the cell population can be CD137⁺ cells. Less than 25 percent of the γδ TCR⁺ cells of the cell population can be PD-1⁺ cells. From 5 to 40 percent of the γδ TCR⁺ cells of the cell population can be BTLA⁺ cells. Greater than 60 percent of the γδ TCR⁺ cells of the cell population can be NKG2D⁺ cells. Greater than 20 percent of the γδ TCR⁺ cells of the cell population can be NKp46⁺ cells. The cells of the cell population can be human cells. The γδ T cells can be tumor infiltrating γδ T cells. The cell population can be a cell population that was produced using a method for producing a cell population comprising γδ T cells as described in any statement or combination of statements from the following paragraph.

The method can comprise (or can consist essentially of or can consist of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69+CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

In another aspect, this document features a method for treating an infection. The method comprises (consists essentially of or consists of) administering a cell population (e.g., an isolated cell population) to a mammal having an infection. The mammal can be a human. The cells of the cell population can be allogenic or autologous to the mammal having the infection. The method can comprise administering αβ T cells to the mammal. The cell population (e.g., isolated cell population) can comprise (or can consist essentially of or can consist of) polyclonal γδ T cells, wherein the population comprises greater than 1×10⁸ γδ T cells. Greater than 85 percent of the CD3⁺ cells the cell population can be γδ TCR⁺ cells. Less than 10 percent of the CD3⁺ cells of the cell population can be αβ TCR⁺ cells. Less than 10 percent of the CD45⁺ cells of the cell population can be NK cells. Greater than 30 percent of the γδ TCR⁺ cells of the cell population can be Vδ1⁺ cells. Less than 60 percent of the γδ TCR⁺ cells of the cell population can be Vδ1⁻Vδ2⁻ cells. Less than 25 percent of the γδ TCR⁺ cells of the cell population can be Vδ2⁺ cells. Greater than 70 percent of the γδ TCR⁺ cells of the cell population can be TEM cells. Less than 25 percent of the γδ TCR⁺ cells of the cell population can be T_{EMRA} cells. Less than 10 percent of the γδ TCR⁺ cells of the cell population can be CD69⁺ CD103⁺ T_{RM} cells. From 1 to 10 percent of the γδ TCR⁺ cells of the cell population can be CD69⁺ CD103⁺ T_{RM} cells. Less than 50 percent of the γδ TCR⁺ cells of the cell population can be CD56⁺ cells. From 1 to 50 percent of the γδ TCR⁺ cells of the cell population can be CD56⁺ cells. From 1 to 40 percent of the γδ TCR⁺ cells of the cell population can be CD137⁺ cells. Less than 25 percent of the γδ TCR⁺ cells of the cell population can be PD-1⁺ cells. From 5 to 40 percent of the γδ TCR⁺ cells of the cell population can be BTLA⁺ cells. Greater than 60 percent of the γδ TCR⁺ cells of the cell population can be NKG2D⁺ cells. Greater than 20 percent of the γδ TCR⁺ cells of the cell population can be NKp46⁺ cells. The cells of the cell population can be human cells. The γδ T cells can be tumor infiltrating γδ T cells. The cell population can be a cell population that was produced using a method for producing a cell population comprising γδ T cells as described in any statement or combination of statements from the following paragraph.

The method can comprise (or can consist essentially of or can consist of) culturing a first cell population comprising γδ T cells in the presence of IL-2, IL-4, and IL-15 for 8 to 21 days to obtain a second cell population, wherein the second cell population comprises at least 10 times more γδ T cells than the first cell population. The γδ T cells can be human cells. The γδ T cells can be tumor infiltrating γδ T cells. The first cell population can be (i) a population of tumor infiltrating γδ T cells obtained from (a) tissue comprising a tumor or (b) healthy tissue that was within 30 mm of a tumor, (ii) a population of γδ T cells obtained from healthy tissue, (iii) a population of γδ T cells obtained from infected tissue, or (iv) a population of γδ T cells obtained from tissue harboring autoimmune T cells. The method can comprise obtaining the first cell population from the tissue comprising the tumor. The method can comprise obtaining the first cell population from the healthy tissue that was within 30 mm of the tumor. The first cell population can be a cell population that was cultured in the presence of 50 international units/mL to 6000 international units/mL of IL-2 and in the absence of IL-4 and IL-15 for 3 to 15 days prior to the culturing in the presence of IL-2, IL-4, and IL-15. The first cell population can be a cell population that was cultured in the presence of 100 international units/mL to 4000 international units/mL of IL-2 and in the absence of IL-4 and IL-15 for 8 to 15 days prior to the culturing in the presence of IL-2, IL-4, and IL-15. The first cell population can be a cell population that was enriched for tumor infiltrating γδ T cells. The first cell population can be a cell population that was enriched for tumor infiltrating γδ T cells via (a) the removal of at least some αβ T cells or (b) the isolation of at least some γδ T cells. The method can comprise removing at least some αβ T cells from a cell population to obtain the first cell population. The removing can comprise positively selecting αβ T cells and removing the positively selected αβ T cells. The method can comprise isolating at least some γδ T cells from a cell population to obtain the first cell population. The isolating can comprise positively selecting γδ T cells and isolating the positively selected γδ T cells. The culturing the first cell population comprising γδ T cells in the presence of IL-2, IL-4, and IL-15 for the 8 to 21 days can comprise culturing the first cell population comprising γδ T cells in the presence of IL-2, IL-4, IL-15, irradiated PBMCs, and an anti-CD3 antibody for the 8 to 21 days. The culturing the first cell population comprising γδ T cells in the presence of IL-2, IL-4, and IL-15 can be for 12 to 16 days. The culturing the first cell population comprising γδ T cells in the presence of IL-2, IL-4, and IL-15 can be for 13 to 15 days. The second cell population can comprise at least 50 times more γδ T cells than the first cell population, at least 100 times more γδ T cells than the first cell population, at least 200 times more γδ T cells than the first cell population, at least 300 times more γδ T cells than the first cell population, or at least 400 times more γδ T cells than the first cell population. The second cell population can comprise greater than 1×10⁸ γδ T cells. The IL-2 can be a human IL-2. The IL-4 can be a human IL-4. The IL-15 can be a human IL-15. Greater than 85 percent of the CD3⁺ cells the second cell population can be γδ TCR⁺ cells. Less than 10 percent of the CD3⁺ cells of the second cell population can be αβ TCR⁺ cells. Less than 10 percent of the CD45⁺ cells of the second cell population can be NK cells. Greater than 30 percent of the γδ TCR⁺ cells of the second cell population can be Vδ1⁺ cells. Less than 60 percent of the γδ TCR⁺ cells of the second cell population can be Vδ1⁻Vδ2⁻ cells. Less than 25 percent of the γδ TCR⁺ cells of the second cell population can be Vδ2⁺ cells. Greater than 70 percent of the γδ TCR⁺ cells of the second cell population can be TEM cells. Less than 25 percent of the γδ TCR⁺ cells of the second cell population can be T_{EMRA} cells. Less than 10 percent of the γδ TCR⁺ cells of the second cell population can be CD69+CD103⁺ T_{RM} cells. From 1 to 10 percent of the γδ TCR⁺ cells of the second cell population can be CD69⁺ CD103⁺ T_{RM} cells. Less than 50 percent of the γδ TCR⁺ cells of the second cell population can be CD56⁺ cells. From 1 to 50 percent of the γδ TCR⁺ cells of the second cell population can be CD56⁺ cells. From 1 to 40 percent of the γδ TCR⁺ cells of the second cell population can be CD137⁺ cells. Less than 25 percent of the γδ TCR⁺ cells of the second cell population can be PD-1⁺ cells. From 5 to 40 percent of the γδ TCR⁺ cells of the second cell population can be BTLA⁺ cells. Greater than 60 percent of the γδ TCR⁺ cells of the second cell population can be NKG2D⁺ cells. Greater than 20 percent of the γδ TCR⁺ cells of the second cell population can be NKp46⁺ cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DESCRIPTION OF THE DRAWINGS

FIGS. 1A-B are photographs of representative pseudomyxoma peritonei (PMP) TIL histology. PMP histology from representative patient tumors are provided using hematoxylin & eosin staining showing focal lymphocytic infiltration restricted to tumor associated stroma. Mucin pools (white) are devoid of lymphocytes.

FIG. 2 is a table providing clinical variables describing PMP patients, whose tumors were used for lymphocyte repertoire sequencing. Representative retrospective tumors were used for histologic analysis and repertoire sequencing of low grade PMP treated with cytoreductive surgery and heated intraperitoneal chemotherapy (CRS-HIPEC). MSS=microsatellite stable; PD-L1=programmed death ligand-1 positivity (which was 0.9% for the selected positive patient).

FIGS. 3A-G. Low grade PMP displayed an elevated BCR IgE fraction associated with TCR Vδ. Following dimer avoidance multiplex polymerase chain reaction (DAM-PCR) and next generation sequencing of RNA isolated from resected formalin fixed paraffin embedded (FFPE) low grade PMP (n=10) tumor tissue, T and B cell receptor CDR3 sequences were constructed with the migec v1.2.9 MiXCR pipeline. FIG. 3A provides representative tree maps of a patient PMP tumor repertoire, where each rounded rectangle represents a unique CDR3, with the size of the rectangle corresponding to the relative frequency of the CDR3 clones across the entire repertoire. Total cohort mean CDR3 expression (FIG. 3B), mean CDR3 amino acid length (FIG. 3C), true entropy repertoire diversity (FIG. 3D), and BCR immunoglobin fraction (FIG. 3E). FIG. 3F provides an IgE fraction comparison between the PMP cohort, healthy donor PBMCs (HD PBMC; n=238), and high grade pancreatic cancer tumors (pancreatic ductal adenocarcinoma; PDAC; n=68). FIG. 3G provides a correlation of TCR Vδ% with BCR IgE fraction in the PMP cohort.

FIGS. 4A-E provides data of PMP repertoire sharing. PMP T and B cell repertoires were compared with healthy donor PBMC repertoires (n=238), identifying shared CDR3s (Public) localized to the Vα, Igκ and Igλ chains (FIG. 4A). FIG. 4B provides the percent breakdown of the n=238 healthy donors with shared uCDR3s within the PMP cohort. FIG. 4C shows the identification of shared CDR3s sequences within PMP cohort that are distinct from public CDR3s, localized to BCR chains. FIG. 4D shows the generational probability of 10 identified IgH sequences (SEQ ID NOs:1-10 from left to right) shared within PMP cohort, with higher probabilities associated with random recombination vs. lower probabilities associated with antigen directed convergent evolution. FIG. 4E shows the BCR immunoglobin fraction of the identified shared 11 IgH PMP CDR3s, composed primarily of IgG, IgE, and IgA. BCR sequences, specifically those of the heavy chain come in 5 different subtypes—IgA, D, E, G, and M; which have different antigen specificities, structural homology, and function. This figure shows that there are public BCRs that are in the PMP repertoire that are shared with the general population (2-3% of IgK and IgL). There are also CDR3 sequences that are shared within the BCR repertoire restricted to patients with PMP. Quantification of the generational probability of these amino acid sequences suggests certain shared sequences are due to random recombination, while others are due to antigen driven recombination, and thus suggestive of convergent evolution of BCR clones against common antigens to PMP tumors. FIG. 4D, does this calculation for the 11 IgH sequences shared within patients in the PMP cohort. FIG. 4E details the immunoglobulin fraction (BCR identity) of the shared PMP IgH sequences, showing they are primary IgG and IgE.

FIGS. 5A-D. γδ TIL sparsely infiltrate peritoneal surface malignancies. FIG. 5A depicts an overview of the study outline. Tumor specimens were harvested prospectively from patients (n=26) with peritoneal surface malignancies undergoing cytoreductive surgery heated intraperitoneal chemotherapy (CRS-HIPEC). Tumor infiltrating lymphocytes were liberated from spatially distinct tumor fragments (n=40 per patient) initially with high concentrations of IL-2 in culture (3,000 IU/mL). RPMI (10% Human AB serum) media and IL-2 were replenished every 3 days in a gas permeable culture flask (G-REX®). On day 11, following spectral cytometry phenotyping, γδ TIL were negatively selected with magnetic bead isolation and rapidly expanded (1×10⁶ cells) with parallel native αβ TIL cultures with a combination of an anti-CD3 antibody (OKT-3 30 ng/mL), IL-2 (3,000 IU/mL), irradiated (30 Gy), allogenic healthy donor PBMCs (1:100; 1×10⁸ cells), and other γ chain cytokines. Spectral cytometry phenotyping of expanded γδ and up TIL was completed on day 25. At the time of tumor fragmentation, the remaining spatially representative tumor fragments were utilized for tumor digestion (Miltenyi Biotech GentleMACS system) and cryopreserved until autologous tumor reactivity assessment with co-culture of expanded TIL and single cell suspension of tumor digest. FIG. 5B provides a comparison of total TIL harvested at day 11 of pre-expansion (pre-REP) culture by peritoneal tumor histology (high grade colon cancer vs low grade (grade 1) appendix cancer). FIG. 5C provides spectral cytometry phenotyping data from day 11 on viable TIL populations (CD56⁺ CD3⁻ NK cells; CD3+γδ TCR⁺ cells; CD3+αβ TCR⁺ CD4⁺ T cells; and CD3+αβ TCR⁺ CD8⁺ T cells). FIG. 5D provides the percentages of γδ Vδ chain subsets as determined on day 11 of pre-expansion (pre-REP) culture by spectral cytometry (CD3+γδ TCR⁺Vδ1⁺, Vδ2+, or Vδ1⁻Vδ2⁻ cells).

FIG. 6 is a table providing prospective CRS-HIPEC patient characteristics. The clinical characteristics of patients with peritoneal surface malignancies undergoing CRS-HIPEC whose tumors were prospectively collected for TIL culture are provided.

FIGS. 7A-E. Peritoneal tumor fragmentation and pre-rapid expansion phenotypic assessment. FIG. 7A provides four sequential photographs of mucinous peritoneal tumor dissection and fragmentation into spatially distinct 2-3 mm³ tumor fragments. On day 11, γδ TCR⁺% and total viable cell counts were compared by histology (FIG. 7B) or prior chemotherapy (FIGS. 7D and 7E). FIG. 7C provides the spectral cytometry gating strategy. CD45⁺ immune cells were selected from live single cells. NK cells (CD3⁻ CD56+) and T cells (CD3+) were selected. T cells were segmented by TCR αβ or γδ positivity. CD4/CD8 or VS1/VS2 populations were identified from selected T cell subsets.

FIGS. 8A-H. γδ TIL display a tissue resident effector memory phenotype with reduced PD-1, but greater NKG2D and CD137 expression compared to αβ TIL. Day 11 peritoneal tumor infiltrating lymphocyte spectral phenotyping comparing αβ and γδ TIL CD8 expression (CD8α⁺, CD8β⁺, or double positivity; FIG. 8A), memory phenotype (CD62L⁺ CD45RO⁻ Naïve; CD62L⁺, CD45RO⁻ Central Memory; CD62L⁻ CD45RO⁻ Effector Memory; CD62L⁻ CD45RO⁻ Effector Memory RA⁺; FIGS. 8B and 8C), tissue resident memory phenotype (CD69⁺, CD103⁺, or double positive; FIG. 8D), activation status (CD2, CD25, CD27, CD56, CD137, or 4-1BB; FIG. 8E), exhaustion status (PD-1, LAG-3, TIGIT, BTLA, or PD-L1; FIG. 8F), and natural cytotoxicity receptor (NCR) expression (NKG2D or NKp46; FIG. 8G). FIG. 8H provides a summary mean expression heat map of CD8, Memory, Activation, Exhaustion, and Natural Cytotoxicity Receptors (NCRs) phenotypes.

FIGS. 9A-E are representative flow diagrams showing αβ and γδ TIL percent positive cells gated by fluorescence minus one (FMO) control and unstimulated PBMC (negative control) on CD8 (FIG. 9A), activation status (CD56 and CD137 or 4-1BB; FIG. 9B), tissue resident memory phenotype (CD69⁺, CD1103⁺, or double positive; FIG. 9C), exhaustion status (PD-1 and BTLA; FIG. 9D), and natural cytotoxicity receptor expression (NKG2D; FIG. 9E).

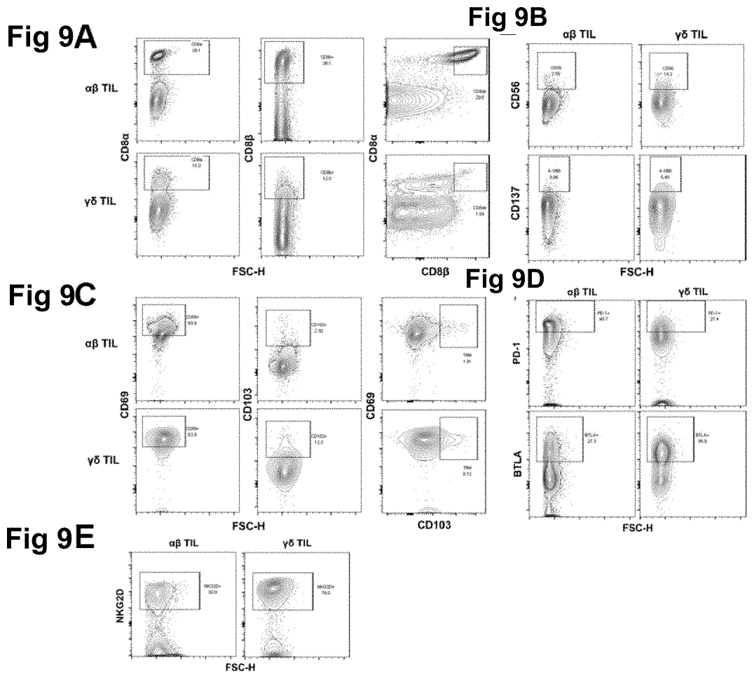

FIGS. 10A-C. Use of IL-4 and IL-15 for rapid expansion of γδ TIL. 1×10⁶ negatively selected γδ TILs or native αβ TILs were expanded for 14 days in culture with an anti-CD3 antibody (OKT-3, 30 ng/mL), irradiated (30 Gy) allogenic PBMC feeders cells (1:100), IL-2 (3,000 IU/mL), RPM 1640 (5% human serum), and the indicated other γ chain cytokines (IL-4 100 ng/mL; IL-7 20 ng/mL; IL-15 70 ng/mL; or combinations thereof) cultured in gas permeable flasks (G-REX®) with cytokines and media replaced every three days. Day 7, 10, and 14 rapid expansion protocol total viable cell counts under individual and varied cytokine combinations were measured (FIG. 10A). The negatively selected γδ TIL population expanded for 14 days with IL-2/IL-4/IL-15 contained minimal NK (CD3⁻ CD56⁺) and αβ T (CD3⁺αβ TCR⁺) cells and γδ T cells (CD3⁺γδ TCR⁺) that were primarily of Vδ1⁺ or Vδ1⁻δ2⁻ cells as assessed by spectral cytometry (FIG. 10B). Average change in absolute percent positive cells between day 11 phenotyping (i.e., pre-expansion) and day 14 post-expansion (i.e., total day 25) of αβ TIL (expanded using an anti-CD3 antibody (OKT-3, 30 ng/mL), irradiated (30 Gy) allogenic PBMC feeders cells (1:100), IL-2 (3,000 IU/mL), and RPM 1640 (5% human serum) and γδ TIL (expanded using a combination of IL-2/IL-4/IL-15) are shown in FIG. 10C. Statistics indicate significant change from day 11 to day 25 of culture as a result of the IL-2/IL-4/IL-15 expansion.

FIGS. 11A-D. Fold expansion and phenotyping of rapidly expanded γδ and up TIL. FIG. 11A shows the fold expansion of negatively selected γδ TIL (e.g., negatively selected by means of αβ TCR depletion) and native αβ TIL following 14 days in culture with the indicated combinations of different cytokines. FIG. 11B provides representative flow plots of post-expansion, negatively selected γδ TIL expanded in culture for 14 days in IL-2, IL-4, and IL-15. FIG. 11C shows the phenotypes of the cells present in the post-IL-2 only expansion of native αβ TIL population. FIG. 11D provides a summary mean expression heat map of memory markers, activation markers, exhaustion markers, and natural cytotoxicity receptors (NCRs) in IL-2 expanded, positively selected αβ TIL and in IL-2/IL-4/IL-15 expanded, negatively selected γδ TIL following 14 days of expansion. Statistics indicate significant difference between γδ and αβ TIL at day 25 of culture as a result of the IL-2/IL-4/IL-15 expansion.

FIGS. 12A-D. MHC independent, γδ TCR mediated autologous tumor recognition. TIL were thawed and rested overnight in IL-2 (3,000 IU/mL) media prior to washing twice in PBS and co-culture. Autologous tumor reactivity was assessed by co-culturing 1×10⁵ of 14 day rapidly expanded αβ TIL (IL-2 expanded, native αβ TIL) or 14 day rapidly expanded γδ TIL (IL-2/IL-4/IL-15 expanded, negatively selected γδ TIL) with 1×10⁵ tumor digest cells in a 96 well plate for 24 hours in cytokine free media. IFNγ production was assessed in culture supernatants by ELISA. FIG. 12A shows IFNγ production of the expanded αβ or γδ TIL following non-specific CD3/CD28 stimulation (Dynabeads, positive control), following co-culture with autologous PBMCs (1×10⁵ cells, negative control), or following co-culture with tumor digest. MHC unrestricted TIL reactivity of expanded γδ TIL and αβ TIL was assessed with the K562 leukemia cancer cell line and a series of colon cancer cell lines (HCT116, RKO, SW480, and SW80) passaged twice prior to co-culture (FIG. 12B). In a subset of patients, γδ TIL were cultured with autologous tumor digests in the presence of blocking antibodies (isotype control mouse IgG 10 μg/mL, anti-MHC-1 (W6/32 10 μg/mL), anti-γδ TCR (7A5 3 μg/mL), or anti-NKG2D (1D11, 10 μg/mL)) (FIG. 12C). FIG. 12D shows the correlation of IFNγ production with the percentage of Vδ1 γδ TIL post-expansion.

FIG. 13 is a table showing cancer cell line NKG2D ligand expression. Tested cancer cell line natural killer receptor ligand mRNA Z-scores were queried from the Cancer Line Encyclopedia. Cell lines with stable or upregulation of MICA and MICB enable γδ TIL recognition.

FIG. 14 is a table describing the clinical characteristics of patients with resected melanoma whose tumor specimens were utilized for tumor digestion and TIL expansion.

FIGS. 15A-E. High dose IL-2 expands the highest number of γδ TCR⁺ TIL during pre-rapid expansion protocol (pre-REP). Cryopreserved melanoma tumor digests (n=15) were thawed and plated (5×10⁶ cells/well) in G-REX® culture wells along with complete media and high dose IL-2 (3,000 IU/mL, n=15, black), γ-chain cytokine combination (IL-2 3,0000 IU/mL, IL-4 100 ng/mL, IL-15 70 ng/mL, n=15, red), or γ-chain cytokine combination plus anti-CD137 mAb (Urelumab, 10 μg/mL, n=10, black and white). Cultures were replenished with cytokine or antibody on Day 4 and Day 8, with 50% of the media replaced on day 8. On Day 11, expanded TIL were harvested through a 70-μm filter, counted (A, B), and analyzed by spectral cytometry for TIL populations (B-E).

FIGS. 16A-G. Vδ1 TIL are associated with a pan cancer survival benefit. FIG. 16A provides the mean expression (Log transcripts per million (TPM)) of γδ TIL subsets (TRDV1, TRDV2, and TRDV3 genes) and αβ TIL (TRBC2 Beta Chain 2 Constant Region) in the 20 most common primary solid tumor types as analyzed by the GEPIA2 tool of the cancer genome atlas (TCGA) database of bulk RNA sequencing of primary tumors. FIGS. 16B-G provide Kaplan Meier survival analyses by normalized (ACTB beta actin) TRDV1 expression above (high) or below (low) the median for selected tumor types where autologous TIL therapy are tested (SKCM=Cutaneous Melanoma; HNSC=Head & Neck Squamous Cell Carcinoma; LUAD+LUSC=Lung Adenocarcinoma and Lung Squamous Cell Carcinoma; BRCA=Breast Carcinoma; and CESC=Cervical Squamous Cell Carcinoma and Endocervical Adenocarcinoma). Log rank P values are displayed along with 95 CI of survival estimates.

FIGS. 17A-G. Vδ1 survival benefit in additional primary cancers. Full cohort TRDV1 tumor (T) and normal (N) tissue expression are plotted for tumor types with the highest Vδ1 expression: Lung Adenocarcinoma (LUAD), Kidney Renal Cell Carcinoma (KIRC), Breast Carcinoma (BRCA), and Cervical Squamous Cell Carcinoma and Endocervical Adenocarcinoma (CESC) (FIG. 17A). FIGS. 17B-G provide Kaplan Meier survival analyses by normalized (ACTB beta actin) TRDV1 expression above (high) or below (low) the median for additional selected tumor types. Log rank P values are displayed along with 95 CI of survival estimates. TCGA=Full Cancer Genome Atlas Tumors; GBM=Glioblastoma; HNSC=Head & Neck Squamous Cell Carcinoma; SKCM=Cutaneous Melanoma; ESCA=Esophageal Carcinoma; LUAD=Lung Adenocarcinoma; LUSC=Lung Squamous cell carcinoma; BRCA=Breast Carcinoma; MESO=Mesothelioma; LIHC=Liver Hepatocellular Carcinoma; STAD=Stomach Adenocarcinoma; PAAD=Pancreatic Ductal Adenocarcinoma; KIRC=Kidney Renal Cell Carcinoma; BLCA=Bladder Urothelial Carcinoma; COAD=Colorectal Adenocarcinoma; READ=Rectal Adenocarcinoma; OV=Ovarian serous cystadenocarcinoma; UCEC=Uterine Corpus Endometrial Carcinoma; CESC=Cervical squamous cell carcinoma and endocervical adenocarcinoma; PRAD=Prostate adenocarcinoma; and SARC=Sarcoma.

FIGS. 18A-S. Correlation of TRDV1 and TRBC2 genes in the 20 most common primary solid tumor types as analyzed by GEPIA2 tool of the cancer genome atlas (TCGA) database of bulk RNA sequencing of primary tumors. Pearson correlation and p value were reported. TCGA abbreviations of FIG. 17 were used. All correlations were significant.

FIGS. 19A-H. No Vδ1 survival benefit in certain primary cancers. FIGS. 19A-H provide Kaplan Meier survival analyses by normalized (ACTB beta actin) TRDV1 expression above (high) or below (low) the median for selected tumor types. Log rank P values were displayed along with 95 CI of survival estimates.

### DETAILED DESCRIPTION

This document provides methods and materials for expanding tumor infiltrating γδ T cells (e.g., tumor infiltrating γδ T cells) in culture. For example, this document provides methods and materials for expanding tumor infiltrating γδ T cells obtained from tissue (e.g., a tumor sample) to obtain large numbers (e.g., greater than 1×10⁷, greater than 1×10⁸, greater than 5×10⁸, or greater than 1×10⁹) of tumor infiltrating γδ T cells (e.g., tumor infiltrating γδ T cells that are predominantly Vδ1⁺) than can be permissible for therapeutic use.

As described herein, tissue containing a tumor (or healthy tissue that is within 30 mm of a tumor, or healthy tissue that is within 20 mm of a tumor, or healthy tissue that is within 10 mm of a tumor) can contain tumor infiltrating γδ T cells and can be obtained from a mammal (e.g., a human cancer patient). In some cases, one or more lymph nodes adjacent to a tumor and/or one or more tumor draining lymph nodes can contain tumor infiltrating γδ T cells and can be obtained from a mammal (e.g., a human cancer patient). For example, lung tissue containing a lung tumor (or healthy lung tissue that is within 30 mm (e.g., within 20 mm or within 10 mm) of a lung tumor or a tumor draining lymph node of a lung tumor) can be obtained from a mammal (e.g., a human lung cancer patient) and used as a source of tumor infiltrating γδ T cells. In another example, skin tissue containing a skin tumor (or healthy skin tissue that is within 30 mm (e.g., within 20 mm or within 10 mm) of a skin tumor or a tumor draining lymph node of a skin tumor) can be obtained from a mammal (e.g., a human skin cancer patient) and used as a source of tumor infiltrating γδ T cells. Other examples of tissues that can be obtained and used as described herein include, without limitation, tissue containing a glioblastoma (or healthy tissue within 30 mm (e.g., within 20 mm or within 10 mm) of a glioblastoma), tissue containing a head & neck squamous cell carcinoma (or healthy tissue within 30 mm (e.g., within 20 mm or within 10 mm) of a head & neck squamous cell carcinoma), tissue containing a cutaneous melanoma (or healthy tissue within 30 mm (e.g., within 20 mm or within 10 mm) of a cutaneous melanoma), tissue containing a lung adenocarcinoma (or healthy tissue within 30 mm (e.g., within 20 mm or within 10 mm) of a lung adenocarcinoma), tissue containing a lung squamous cell carcinoma (or healthy tissue within 30 mm (e.g., within 20 mm or within 10 mm) of a lung squamous cell carcinoma), tissue containing a breast carcinoma (or healthy tissue within 30 mm (e.g., within 20 mm or within 10 mm) of a breast carcinoma), tissue containing a mesothelioma (or healthy tissue within 30 mm (e.g., within 20 mm or within 10 mm) of a mesothelioma), tissue containing a liver hepatocellular carcinoma (or healthy tissue within 30 mm (e.g., within 20 mm or within 10 mm) of a liver hepatocellular carcinoma), tissue containing a pancreatic ductal adenocarcinoma (or healthy tissue within 30 mm (e.g., within 20 mm or within 10 mm) of a pancreatic ductal adenocarcinoma), tissue containing a kidney renal cell carcinoma (or healthy tissue within 30 mm (e.g., within 20 mm or within 10 mm) of a kidney renal cell carcinoma), tissue containing a bladder urothelial carcinoma (or healthy tissue within 30 mm (e.g., within 20 mm or within 10 mm) of a bladder urothelial carcinoma), tissue containing a cervical squamous cell carcinoma and endocervical adenocarcinoma (or healthy tissue within 30 mm (e.g., within 20 mm or within 10 mm) of a cervical squamous cell carcinoma and endocervical adenocarcinoma), tissue containing a lymph node metastases, tissue containing a peritoneum tumor (or healthy tissue within 30 mm (e.g., within 20 mm or within 10 mm) of a peritoneum tumor), tissue containing a bone tumor (or healthy tissue within 30 mm (e.g., within 20 mm or within 10 mm) of a bone tumor), tissue containing an endocrine gland tumor (or healthy tissue within 30 mm (e.g., within 20 mm or within 10 mm) of an endocrine gland tumor), tissue containing a reproductive organ tumor (or healthy tissue within 30 mm (e.g., within 20 mm or within 10 mm) of a reproductive organ tumor), or tissue containing a brain (or healthy tissue within 30 mm (e.g., within 20 mm or within 10 mm) of a brain tumor).

Once a tissue is obtained, the tissue can be cultured in a manner that promotes the isolation of tumor infiltrating γδ T cells from the tissue. For example, one or more pieces (e.g., 2-3 mm³ pieces) of the tissue can be cultured in the presence of IL-2 for 5 to 15 days (e.g., 6 to 15 days, 7 to 15 days, 8 to 15 days, 9 to 15 days, 9 to 13 days, 10 to 12 days, 7 to 10 days, or 8 to 14 days). In some cases, the one or more pieces (e.g., 2-3 mm³ pieces) of the tissue can be cultured in a gas permeable rapid expansion flask. Any appropriate concentration of IL-2 can be used to promote the isolation of tumor infiltrating γδ T cells from the tissue. For example, from about 50 international units (IU) to about 6000 IU (e.g., from about 100 IU to about 6000 IU, from about 500 IU to about 6000 IU, from about 1000 IU to about 6000 IU, from about 1500 IU to about 6000 IU, from about 2000 IU to about 6000 IU, from about 2500 IU to about 6000 IU, from about 3000 IU to about 6000 IU, from about 3500 IU to about 6000 IU, from about 2500 IU to about 4000 IU, or from about 2500 IU to about 3500 IU) of IL-2 per mL of culture medium can be used.

In some cases, tissue (e.g., tumor tissue) can be mechanically and/or enzymatically digested, and a single cell tumor digest suspension can be cultured for a period of time or γδ T cells can be directly isolated at this time.

After culturing tissue containing tumor infiltrating γδ T cells with IL-2 for 5 to 15 days (e.g., 6 to 15 days, 7 to 15 days, 8 to 15 days, 9 to 15 days, 9 to 13 days, 10 to 12 days, 7 to 10 days, or 8 to 14 days), a cell population that exited the tissue can be harvested. In some cases, the harvested cell population can include tumor infiltrating αβ T cells and tumor infiltrating γδ T cells. In some cases, the harvested cell population can include a greater number of tumor infiltrating up T cells than the number of tumor infiltrating γδ T cells. In some cases, an anti-αβ TCR antibody, an anti-CD28 antibody, an anti-4-1BBL antibody, an anti-GITR antibody, an anti-CD27 antibody, or a combination thereof can be used to promote a cell population that is enriched for γδ T cells from the harvested cell population.

Once the harvested cell population is obtained, an optional enrichment for γδ T cells can be performed. For example, magnetic beads containing an anti-αβ TCR antibody can be used in a negative selection process to remove αβ T cells from the harvested cell population to obtain a cell population enriched for γδ T cells. In some cases, an anti-TCR γδ antibody, an anti-Vδ1 antibody, an anti-NKG2D antibody, or a combination thereof can be used in a positive selection process to isolate γδ T cells from the harvested cell population to obtain a cell population enriched for γδ T cells.

Briefly, when using antibodies to remove non-γδ T cells (e.g., αβ T cells) from or to isolate γδ T cells from the harvested cell population to obtain a cell population enriched for γδ T cells, the antibodies can be biotinylated and can be attached to a magnetic substrate (e.g., a magnetic bead) via streptavidin. In some cases, flow activated cell sorting (FACS) can be used to remove non-γδ T cells (e.g., αβ T cells) from or to isolate γδ T cells from the harvested cell population to obtain a cell population enriched for γδ T cells.

In some cases, the harvested cell population (or a portion thereof) can be used for expanding the number of γδ T cells without the optional enrichment step.

Once the harvested cell population (with or without the optional enrichment for γδ T cells) is obtained, the harvested cell population (or a portion thereof) can be used to perform an expansion step that increases the number of γδ T cells present. In some cases, this expansion step can increase the starting number of γδ T cells present in the starting cell population to a number of γδ T cells present in the resulting cell population that is from 10 to 1000 fold greater (e.g., 10 to 600 fold, 20 to 600 fold, 30 to 600 fold, 40 to 600 fold, 50 to 600 fold, 75 to 600 fold, 100 to 600 fold, 200 to 1000 fold, 250 to 1000 fold, 300 to 1000 fold, 350 to 1000 fold, 400 to 1000 fold, 450 to 1000 fold, 500 to 1000 fold, 200 to 1000 fold, 250 to 1000 fold, 300 to 51000 00 fold, 350 to 1000 fold, 400 to 1000 fold, or 450 to 1000 fold) greater than that starting number. In some cases, this expansion step can increase the starting number of γδ T cells present in the starting cell population to a number of γδ T cells present in the resulting cell population that is more than 200 fold greater (e.g., more than 250 fold greater, more than 300 fold greater, more than 350 fold greater, more than 400 fold greater, or more than 450 fold greater) than that starting number. In some cases, this expansion step can expand the starting number of γδ T cells present in the starting cell population to a number of γδ T cells present in the resulting cell population that is 200 to 600 fold (e.g., 200 to 600 fold, 250 to 600 fold, 300 to 600 fold, 350 to 600 fold, 400 to 600 fold, 450 to 600 fold, 500 to 600 fold, 200 to 550 fold, 250 to 550 fold, 300 to 550 fold, 350 to 550 fold, 400 to 550 fold, 450 to 550 fold, 500 to 550 fold, 200 to 500 fold, 250 to 500 fold, 300 to 500 fold, 350 to 500 fold, 400 to 500 fold, or 450 to 500 fold) greater than that starting number. In some cases, this expansion step can increase the starting number of γδ T cells present in the starting cell population to a number of γδ T cells present in the resulting cell population that is greater than 25 percent (e.g., greater than 50 percent, greater than 75 percent, or greater than 95 percent) enriched in γδ T cells.

Any appropriate method can be used to promote the expansion of γδ T cells of a harvested cell population (or a portion thereof) or a harvested, γδ T cell-enriched cell population (or a portion thereof). For example, a harvested cell population (with or without the optional enrichment for γδ T cells) or a portion thereof can be cultured in the presence of IL-2, IL-4, and IL-15 to promote the expansion of γδ T cells. The amount of IL-2 can be from about 50 IU to about 6000 IU (e.g., from about 100 IU to about 6000 IU, from about 500 IU to about 6000 IU, from about 1000 IU to about 6000 IU, from about 1500 IU to about 6000 IU, from about 2000 IU to about 6000 IU, from about 2500 IU to about 6000 IU, from about 3000 IU to about 6000 IU, from about 3500 IU to about 6000 IU, from about 2500 IU to about 4000 IU, or from about 2500 IU to about 3500 IU) of IL-2/mL of culture medium. The amount of IL-4 can be from about 10 ng to about 200 ng (e.g., from about 20 ng to about 200 ng, from about 50 ng to about 200 ng, from about 75 ng to about 200 ng, from about 10 ng to about 150 ng, from about 10 ng to about 100 ng, from about 50 ng to about 150 ng, or from about 90 ng to about 110 ng) of IL-4/mL of culture medium. The amount of IL-15 can be from about 10 ng to about 200 ng (e.g., from about 20 ng to about 200 ng, from about 50 ng to about 200 ng, from about 75 ng to about 200 ng, from about 10 ng to about 150 ng, from about 10 ng to about 100 ng, from about 50 ng to about 150 ng, from about 50 ng to about 90 ng, or from about 60 ng to about 90 ng) of IL-15/mL of culture medium.

In some cases, a harvested cell population (with or without the optional enrichment for γδ T cells) or a portion thereof can be cultured in the presence of IL-2, IL-4, and IL-15 with the optional inclusion of IL-7 and/or IL-21. When optionally including IL-7, amount of IL-7 can be from about 10 ng to about 200 ng (e.g., from about 20 ng to about 200 ng, from about 50 ng to about 200 ng, from about 75 ng to about 200 ng, from about 10 ng to about 150 ng, from about 10 ng to about 100 ng, from about 50 ng to about 150 ng, or from about 90 ng to about 110 ng) of IL-7/mL of culture medium. When optionally including IL-21, amount of IL-21 can be from about 10 ng to about 200 ng (e.g., from about 20 ng to about 200 ng, from about 50 ng to about 200 ng, from about 75 ng to about 200 ng, from about 10 ng to about 150 ng, from about 10 ng to about 100 ng, from about 50 ng to about 150 ng, or from about 90 ng to about 110 ng) of IL-21/mL of culture medium.

Any appropriate IL-2, IL-4, and IL-15 (and optionally included IL-7 and/or IL-21) can be used to expand γδ T cells as described herein. For example, when expanding human γδ T cells, then human IL-2, human IL-4, and human IL-15 can be used to expand the human γδ T cells. In another example, when expanding horse γδ T cells, then horse IL-2, horse IL-4, and horse IL-15 can be used to expand the horse γδ T cells. In another example, when expanding monkey γδ T cells, then monkey IL-2, monkey IL-4, and monkey IL-15 can be used to expand the monkey γδ T cells. In another example, when expanding dog γδ T cells, then dog IL-2, dog IL-4, and dog IL-15 can be used to expand the dog γδ T cells.

The harvested cell population (with or without the optional enrichment for γδ T cells) or a portion thereof can be cultured in the presence of IL-2, IL-4, and IL-15 for any appropriate length of time to promote the expansion of γδ T cells. For example, a harvested cell population (with or without the optional enrichment for γδ T cells) or a portion thereof can be culture in the presence of IL-2, IL-4, and IL-15 for 8 to 21 days (e.g., 10 to 21 days, 12 to 21 days, 14 to 21 days, 8 to 18 days, 8 to 16 days, 8 to 14 days, 10 to 20 days, 10 to 18 days, 12 to 18 days, 10 to 16 days, 12 to 16 days, or 13 to 15 days). In some cases, the IL-2, IL-4, and IL-15 in the culture can be replenished every 3 days, every 4-6 days, or every 2-3 days.

In some cases, the culture containing IL-2, IL-4, and IL-15 and being used to expand the number of γδ T cells can contain one or more additional agents. For example, in addition to IL-2, IL-4, and IL-15, the culture can contain anti-CD3 antibodies (e.g., soluble and/or immobilized anti-CD3 antibodies), anti-CD28 antibodies (e.g., soluble and/or immobilized anti-CD28 antibodies), irradiated PBMCs (e.g., irradiated PBMCs that are autologous to the mammal to be treated with the expanded γδ T cells), agonistic anti-γδ TCR antibodies (e.g., soluble and/or immobilized anti-γδ TCR antibodies such as Vδ1 antibodies; about 1 μg/mL; see, e.g., Zhou et al., Cell Mol. Immunol., 9(1):34-44 (2012)), anti-4-1BB antibodies (e.g., soluble and/or immobilized anti-4-1BB antibodies such as Urelumab; about 10 μg/mL; see, e.g., Sakellariou-Thompson et al., Clin. Cancer Res., 23(23):7263-7275 (2017)), anti-TIGIT antibodies (e.g., soluble and/or immobilized anti-TIGIT antibodies; 1 μg/mL; see, e.g., Chauvin et al., J Clin. Invest., 125(5):2046-58 (2015)), high glucose (e.g., from 5 mM to 25 mM, from 8 mM to 20 mM, from 8 mM to 12 mM, or from 9 mM to 11 mM of glucose; see, e.g., Lopes et al., Nat. Immunol., 22:179-192 (2021)), irradiated artificial antigen presenting cells (e.g., cloned K562 cells transfected with 4-1BBL, CD86, IL-15/membrane bound IL-15; 1:100 ratio; see, e.g., Deniger et al., Clin. Cancer Res., 20(22):5708-5719 (2014)), PHA (about 1 μg/mL), irradiated EBV transfected B cell lines (1:100 ratio; see, e.g., Ma et al., J Exp. Med., 208(3):491-503 (2011)), anti-OX40 antibodies (e.g., soluble and/or immobilized anti-OX40 antibodies), phosphoinositide 3-kinase (PI 3-kinase) inhibitors (e.g., Idelalisib, Copanlisib, Duvelisib, Alpelisib, or Umbralisib), CDK4/6 inhibitors (e.g., palbociclib, ribociclib, or abemaciclib; see, e.g., Lelliott et al., Cancer Discov., 11(10):2582-2601 (2021)), CBL-B inhibitors (e.g., NX-0255 or NX-1607; see, e.g., Rountree et al., Cancer Res., Jul. 1, 2021 (81) (13 Supplement):1595), STS1 inhibitors (see, e.g., Hwang et al., Exp. Mole. Med., 52:750-761 (2020)), CISH (see, e.g., Palmer et al., J. Exp. Med., 212(12):2095-2113 (2015)), TET2 (see, e.g., Fraietta et al., Nature, 558(7709):307-312 (2018)), or combinations thereof. For example, in addition to IL-2, IL-4, and IL-15, the culture can contain anti-CD3 antibodies (e.g., soluble anti-CD3 antibodies) and irradiated PBMCs. The amount of anti-CD3 antibodies can be from about 0.1 μg to about 1 μg of anti-CD3 antibodies per mL of culture medium. The amount of anti-CD28 antibodies can be from about 500 ng to about 5 μg of anti-CD28 antibodies per mL of culture medium. The amount of irradiated PBMCs can be based on the number of input γδ T cells such that the ratio of γδ T cells:PBMCs is from about 1:25 to about 1:200 (e.g., 1:100).

After expanding the number of γδ T cells in the presence of IL-2, IL-4, and IL-15, the cells can be washed to remove any particular components of the culture medium. For example, after the expansion step is completed, the resulting cell population can be washed to remove any remaining IL-2, IL-4, IL-15, anti-CD3 antibodies, and/or anti-CD28 antibodies, and/or the expanded γδ T cells can be concentrated. In some cases, after the expansion step, the expanded γδ T cells can be cultured in the absence of IL-2, IL-4, and/or IL-15 for any appropriate length of time. For example, after the rapid expansion step, the population of expanded γδ T cells can be cultured in the absence of IL-2, IL-4, and/or IL-15 for 10 to 75 days (e.g., 10 to 60 days, 10 to 50 days, or 10 to 25 days). In some cases, expanded γδ T cells can be obtained from multiple donors (e.g., multiple humans) and pooled to provide a population of γδ T cells for treating one or more patients (e.g., one or more humans).

As described herein, a cell population containing expanded γδ T cells that results from a γδ T cell expansion in the presence of IL-2, IL-4, and IL-15 as described herein can have a particularly desired make up of cells. For example, in some cases, greater than 85 percent (e.g., greater than 90 percent, greater than 91 percent, greater than 92 percent, greater than 93 percent, greater than 94 percent, greater than 95 percent, greater than 96 percent, greater than 97 percent, greater than 98 percent, or greater than 99 percent) of the CD3⁺ cells of a population provided herein can be γδ TCR⁺ cells. In some cases, less than 10 percent (e.g., less than 9 percent, less than 8 percent, less than 7 percent, less than 6 percent, less than 5 percent, less than 4 percent, less than 3 percent, less than 2 percent, or less than 1 percent) of the CD3⁺ cells of a population provided herein can be αβ TCR⁺ cells. In some cases, less than 10 percent (e.g., less than 9 percent, less than 8 percent, less than 7 percent, less than 6 percent, less than 5 percent, less than 4 percent, less than 3 percent, less than 2 percent, or less than 1 percent) of the CD45⁺ cells of a population provided herein can be NK cells.

In some cases, a cell population containing expanded γδ T cells that results from a γδ T cell expansion in the presence of IL-2, IL-4, and IL-15 as described herein can vary and can include not only αβ T cells, but also phenotypic NKT, NK and B cells in various proportions.

In some cases, greater than 85 percent (e.g., greater than 90 percent, greater than 91 percent, greater than 92 percent, greater than 93 percent, greater than 94 percent, greater than 95 percent, greater than 96 percent, greater than 97 percent, greater than 98 percent, or greater than 99 percent) of the CD3⁺ cells of a population provided herein can be γδ TCR⁺ cells and less than 10 percent (e.g., less than 9 percent, less than 8 percent, less than 7 percent, less than 6 percent, less than 5 percent, less than 4 percent, less than 3 percent, less than 2 percent, or less than 1 percent) of the CD3⁺ cells of that population can be αβ TCR⁺ cells.

In some cases, greater than 85 percent (e.g., greater than 90 percent, greater than 91 percent, greater than 92 percent, greater than 93 percent, greater than 94 percent, greater than 95 percent, greater than 96 percent, greater than 97 percent, greater than 98 percent, or greater than 99 percent) of the CD3⁺ cells of a population provided herein can be γδ TCR⁺ cells and less than 10 percent (e.g., less than 9 percent, less than 8 percent, less than 7 percent, less than 6 percent, less than 5 percent, less than 4 percent, less than 3 percent, less than 2 percent, or less than 1 percent) of the CD45⁺ cells of that population can be NK cells.

In some cases, greater than 85 percent (e.g., greater than 90 percent, greater than 91 percent, greater than 92 percent, greater than 93 percent, greater than 94 percent, greater than 95 percent, greater than 96 percent, greater than 97 percent, greater than 98 percent, or greater than 99 percent) of the CD3⁺ cells of a population provided herein can be γδ TCR⁺ cells, less than 10 percent (e.g., less than 9 percent, less than 8 percent, less than 7 percent, less than 6 percent, less than 5 percent, less than 4 percent, less than 3 percent, less than 2 percent, or less than 1 percent) of the CD3⁺ cells of that population can be αβ TCR⁺ cells, and less than 10 percent (e.g., less than 9 percent, less than 8 percent, less than 7 percent, less than 6 percent, less than 5 percent, less than 4 percent, less than 3 percent, less than 2 percent, or less than 1 percent) of the CD45⁺ cells of that population can be NK cells.

In some cases, greater than 30 percent (e.g., greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be Vδ1⁺ cells.

In some cases, less than 60 percent (e.g., less than 55 percent, less than 50 percent, less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of a population provided herein can be VDδ1⁻ Vδ2⁻ cells.

In some cases, less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of a population provided herein can be Vδ2⁺ cells.

In some cases, greater than 30 percent (e.g., greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be Vδ1⁺ cells and less than 60 percent (e.g., less than 55 percent, less than 50 percent, less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of that population can be VDδ1⁻Vδ2⁻ cells.

In some cases, greater than 30 percent (e.g., greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be Vδ1⁺ cells and less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be Vδ2⁺ cells.

In some cases, greater than 30 percent (e.g., greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be Vδ1⁺ cells, less than 60 percent (e.g., less than 55 percent, less than 50 percent, less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of that population can be VDδ1⁻Vδ2⁻ cells, and less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be Vδ2⁺ cells.

In some cases, greater than 70 percent (e.g., greater than 75 percent, greater than 80 percent, greater than 85 percent, greater than 90 percent, or greater than 95 percent) of the γδ TCR⁺ cells of a population provided herein can be T_{EM} cells.

In some cases, less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of a population provided herein can be T_{EMRA} cells.

In some cases, greater than 70 percent (e.g., greater than 75 percent, greater than 80 percent, greater than 85 percent, greater than 90 percent, or greater than 95 percent) of the γδ TCR⁺ cells of a population provided herein can be TEM cells and less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be T_{EMRA} cells.

In some cases, a population provided herein can have a higher percentage (e.g., a percentage that is 2 to 40 percentage points higher, 5 to 40 percentage points higher, 10 to 40 percentage points higher, 15 to 40 percentage points higher, 20 to 40 percentage points higher, 5 to 35 percentage points higher, 5 to 30 percentage points higher, 5 to 25 percentage points higher, 5 to 20 percentage points higher, 5 to 15 percentage points higher, or 5 to 10 percentage points higher) of γδ TCR⁺ TEM cells following cell expansion in the presence of IL-2, IL-4, and IL-15 than the starting population had before cell expansion in the presence of IL-2, IL-4, and IL-15.

In some cases, a population provided herein can have a lower percentage (e.g., a percentage that is 2 to 30 percentage points lower, 5 to 30 percentage points lower, 10 to 30 percentage points lower, 15 to 30 percentage points lower, 20 to 30 percentage points lower, 5 to 25 percentage points lower, 5 to 20 percentage points lower, 5 to 15 percentage points lower, or 5 to 10 percentage points lower) of γδ TCR⁺ T_{EMRA} cells following cell expansion in the presence of IL-2, IL-4, and IL-15 than the starting population had before cell expansion in the presence of IL-2, IL-4, and IL-15.

In some cases, a population provided herein can have a higher percentage (e.g., a percentage that is 2 to 40 percentage points higher, 5 to 40 percentage points higher, 10 to 40 percentage points higher, 15 to 40 percentage points higher, 20 to 40 percentage points higher, 5 to 35 percentage points higher, 5 to 30 percentage points higher, 5 to 25 percentage points higher, 5 to 20 percentage points higher, 5 to 15 percentage points higher, or 5 to 10 percentage points higher) of γδ TCR⁺ TEM cells and a lower percentage (e.g., a percentage that is 2 to 30 percentage points lower, 5 to 30 percentage points lower, 10 to 30 percentage points lower, 15 to 30 percentage points lower, 20 to 30 percentage points lower, 5 to 25 percentage points lower, 5 to 20 percentage points lower, 5 to 15 percentage points lower, or 5 to 10 percentage points lower) of γδ TCR⁺ T_{EMRA} cells following cell expansion in the presence of IL-2, IL-4, and IL-15 than the starting population had before cell expansion in the presence of IL-2, IL-4, and IL-15.

In some cases, less than 10 percent (e.g., less than 9 percent, less than 8 percent, less than 7 percent, less than 6 percent, less than 5 percent, less than 4 percent, less than 3 percent, less than 2 percent, or less than 1 percent) of the γδ TCR⁺ cells of a population provided herein can be CD69⁺ CD103⁺ T_{RM}. In some cases, from 1 to 10 percent (e.g., from 1 to 9 percent, from 1 to 8 percent, from 1 to 7 percent, from 1 to 6 percent, from 1 to 5 percent, from 1 to 4 percent, from 2 to 10 percent, from 3 to 10 percent, from 4 to 10 percent, from 5 to 10 percent, from 6 to 10 percent, from 2 to 8 percent, from 2 to 6 percent, from 4 to 8 percent, or from 4 to 6 percent) of the γδ TCR⁺ cells of a population provided herein can be CD69⁺ CD103⁺ T_{RM} cells.

In some cases, less than 50 percent (e.g., less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of a population provided herein can be CD56⁺ cells. In some cases, from 1 to 50 percent (e.g., from 1 to 45 percent, from 1 to 40 percent, from 1 to 35 percent, from 1 to 30 percent, from 1 to 25 percent, from 1 to 20 percent, from 5 to 50 percent, from 10 to 50 percent, from 15 to 50 percent, from 20 to 50 percent, from 10 to 40 percent, from 15 to 35 percent, or from 20 to 30 percent) of the γδ TCR⁺ cells of a population provided herein can be CD56⁺ cells.

In some cases, from 1 to 40 percent (e.g., from 1 to 35 percent, from 1 to 30 percent, from 1 to 25 percent, from 1 to 20 percent, from 1 to 15 percent, from 1 to 10 percent, from 5 to 40 percent, from 10 to 40 percent, from 15 to 40 percent, from 20 to 40 percent, from 5 to 35 percent, from 10 to 30 percent, or from 15 to 25 percent) of the γδ TCR⁺ cells of a population provided herein can be CD137⁺ cells.

In some cases, a population provided herein can have a higher percentage (e.g., a percentage that is 2 to 50 percentage points higher, 5 to 50 percentage points higher, 2 to 40 percentage points higher, 5 to 40 percentage points higher, 10 to 40 percentage points higher, 15 to 40 percentage points higher, 20 to 40 percentage points higher, 5 to 35 percentage points higher, 5 to 30 percentage points higher, 5 to 25 percentage points higher, 5 to 20 percentage points higher, 5 to 15 percentage points higher, or 5 to 10 percentage points higher) of CD137⁺γδ TCR⁺ cells following cell expansion in the presence of IL-2, IL-4, and IL-15 than the starting population had before cell expansion in the presence of IL-2, IL-4, and IL-15.

In some cases, less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of a population provided herein can be PD-1⁺ cells.

In some cases, a population provided herein can have a lower percentage (e.g., a percentage that is 5 to 90 percentage points lower, 5 to 80 percentage points lower, 5 to 75 percentage points lower, 5 to 70 percentage points lower, 5 to 75 percentage points lower, 5 to 70 percentage points lower, 5 to 65 percentage points lower, 5 to 60 percentage points lower, 5 to 55 percentage points lower, 5 to 50 percentage points lower, 5 to 45 percentage points lower, 5 to 40 percentage points lower, 5 to 35 percentage points lower, 5 to 30 percentage points lower, 5 to 25 percentage points lower, 5 to 20 percentage points lower, 5 to 15 percentage points lower, 5 to 10 percentage points lower, 10 to 90 percentage points lower, 10 to 80 percentage points lower, 10 to 75 percentage points lower, 10 to 70 percentage points lower, 10 to 75 percentage points lower, 10 to 70 percentage points lower, 10 to 65 percentage points lower, 10 to 60 percentage points lower, 10 to 55 percentage points lower, 10 to 50 percentage points lower, 10 to 45 percentage points lower, 10 to 40 percentage points lower, 10 to 35 percentage points lower, 10 to 30 percentage points lower, 10 to 25 percentage points lower, 10 to 20 percentage points lower, 10 to 15 percentage points lower, 25 to 90 percentage points lower, 25 to 80 percentage points lower, 25 to 75 percentage points lower, 25 to 70 percentage points lower, 25 to 75 percentage points lower, 25 to 70 percentage points lower, 25 to 65 percentage points lower, 25 to 60 percentage points lower, 25 to 55 percentage points lower, 25 to 50 percentage points lower, 25 to 45 percentage points lower, 25 to 40 percentage points lower, 25 to 35 percentage points lower, or 25 to 30 percentage points lower) of PD-1⁺γδ TCR⁺ cells following cell expansion in the presence of IL-2, IL-4, and IL-15 than the starting population had before cell expansion in the presence of IL-2, IL-4, and IL-15.

In some cases, from 5 to 40 percent (e.g., from 5 to 35 percent, from 5 to 30 percent, from 5 to 25 percent, from 5 to 20 percent, from 5 to 15 percent, from 10 to 40 percent, from 10 to 35 percent, from 10 to 30 percent, from 10 to 25 percent, from 10 to 20 percent, or from 15 to 25 percent) of the γδ TCR⁺ cells of a population provided herein can be BTLA⁺ cells.

In some cases, from 5 to 40 percent (e.g., from 5 to 35 percent, from 5 to 30 percent, from 5 to 25 percent, from 5 to 20 percent, from 5 to 15 percent, from 10 to 40 percent, from 10 to 35 percent, from 10 to 30 percent, from 10 to 25 percent, from 10 to 20 percent, or from 15 to 25 percent) of the αβ TCR⁺ cells of a population provided herein can be BTLA⁺ cells.

In some cases, greater than 60 percent (e.g., greater than 65 percent, greater than 70 percent, greater than 75 percent, greater than 80 percent, greater than 85 percent, greater than 90 percent, or greater than 95 percent) of the γδ TCR⁺ cells of a population provided herein can be NKG2D⁺ cells.

In some cases, greater than 20 percent (e.g., greater than 25 percent, greater than 30 percent, greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be NKp46⁺ cells.

In some cases, a population provided herein can have a higher percentage (e.g., a percentage that is 5 to 90 percentage points higher, 5 to 85 percentage points higher, 5 to 80 percentage points higher, 5 to 75 percentage points higher, 5 to 70 percentage points higher, 5 to 65 percentage points higher, 5 to 60 percentage points higher, 5 to 55 percentage points higher, 5 to 50 percentage points higher, 5 to 45 percentage points higher, 5 to 40 percentage points higher, 5 to 35 percentage points higher, 5 to 30 percentage points higher, 5 to 25 percentage points higher, 10 to 90 percentage points higher, 10 to 85 percentage points higher, 10 to 80 percentage points higher, 10 to 75 percentage points higher, 10 to 70 percentage points higher, 10 to 65 percentage points higher, 10 to 60 percentage points higher, 10 to 55 percentage points higher, 10 to 50 percentage points higher, 10 to 45 percentage points higher, 10 to 40 percentage points higher, 10 to 35 percentage points higher, 10 to 30 percentage points higher, 10 to 25 percentage points higher, 15 to 90 percentage points higher, 15 to 85 percentage points higher, 15 to 80 percentage points higher, 15 to 75 percentage points higher, 15 to 70 percentage points higher, 15 to 65 percentage points higher, 15 to 60 percentage points higher, 15 to 55 percentage points higher, 15 to 50 percentage points higher, 15 to 45 percentage points higher, 15 to 40 percentage points higher, 15 to 45 percentage points higher, 15 to 30 percentage points higher, 15 to 25 percentage points higher, or 20 to 40 percentage points higher) of NKp46⁺ cells following cell expansion in the presence of IL-2, IL-4, and IL-15 than the starting population had before cell expansion in the presence of IL-2, IL-4, and IL-15.

In some cases, (a) greater than 85 percent (e.g., greater than 90 percent, greater than 91 percent, greater than 92 percent, greater than 93 percent, greater than 94 percent, greater than 95 percent, greater than 96 percent, greater than 97 percent, greater than 98 percent, or greater than 99 percent) of the CD3⁺ cells of a population provided herein can be γδ TCR⁺ cells, (b) less than 10 percent (e.g., less than 9 percent, less than 8 percent, less than 7 percent, less than 6 percent, less than 5 percent, less than 4 percent, less than 3 percent, less than 2 percent, or less than 1 percent) of the CD3⁺ cells of that population can be αβ TCR⁺ cells, (c) less than 10 percent (e.g., less than 9 percent, less than 8 percent, less than 7 percent, less than 6 percent, less than 5 percent, less than 4 percent, less than 3 percent, less than 2 percent, or less than 1 percent) of the CD45⁺ cells of that population can be NK cells, (d) greater than 30 percent (e.g., greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of a population provided herein can be Vδ1⁺ cells, (e) less than 60 percent (e.g., less than 55 percent, less than 50 percent, less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of that population can be VDδ1⁻Vδ2⁻ cells, (f) less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be Vδ2⁺ cells, (g) greater than 70 percent (e.g., greater than 75 percent, greater than 80 percent, greater than 85 percent, greater than 90 percent, or greater than 95 percent) of the γδ TCR⁺ cells of a population provided herein can be TEM cells, (h) less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be T_{EMRA} cells, (i) less than 10 percent (e.g., less than 9 percent, less than 8 percent, less than 7 percent, less than 6 percent, less than 5 percent, less than 4 percent, less than 3 percent, less than 2 percent, or less than 1 percent) of the γδ TCR⁺ cells of that population can be CD69⁺ CD103⁺ T_{RM} or from 1 to 10 percent (e.g., from 1 to 9 percent, from 1 to 8 percent, from 1 to 7 percent, from 1 to 6 percent, from 1 to 5 percent, from 1 to 4 percent, from 2 to 10 percent, from 3 to 10 percent, from 4 to 10 percent, from 5 to 10 percent, from 6 to 10 percent, from 2 to 8 percent, from 2 to 6 percent, from 4 to 8 percent, or from 4 to 6 percent) of the γδ TCR⁺ cells of that population can be CD69⁺ CD103⁺ T_{RM} cells, (j) less than 50 percent (e.g., less than 45 percent, less than 40 percent, less than 35 percent, less than 30 percent, less than 25 percent, less than 20 percent, less than 15 percent, less than 10 percent, less than 5 percent, or less than 2 percent) of the γδ TCR⁺ cells of that population can be CD56⁺ cells or from 1 to 50 percent (e.g., from 1 to 45 percent, from 1 to 40 percent, from 1 to 35 percent, from 1 to 30 percent, from 1 to 25 percent, from 1 to 20 percent, from 5 to 50 percent, from 10 to 50 percent, from 15 to 50 percent, from 20 to 50 percent, from 10 to 40 percent, from 15 to 35 percent, or from 20 to 30 percent) of the γδ TCR⁺ cells of that population can be CD56⁺ cells, (k) from 1 to 40 percent (e.g., from 1 to 35 percent, from 1 to 30 percent, from 1 to 25 percent, from 1 to 20 percent, from 1 to 15 percent, from 1 to 10 percent, from 5 to 40 percent, from 10 to 40 percent, from 15 to 40 percent, from 20 to 40 percent, from 5 to 35 percent, from 10 to 30 percent, or from 15 to 25 percent) of the γδ TCR⁺ cells of that population can be CD137⁺ cells, (1) less than 25 percent (e.g., less than 20 percent, less than 15 percent, less than 10 percent, less than 5 percent, less than 4 percent, less than 3 percent, less than 2 percent, or less than 1 percent) of the γδ TCR⁺ cells of that population can be PD-1⁺ cells, (m) from 5 to 40 percent (e.g., from 5 to 35 percent, from 5 to 30 percent, from 5 to 25 percent, from 5 to 20 percent, from 5 to 15 percent, from 10 to 40 percent, from 10 to 35 percent, from 10 to 30 percent, from 10 to 25 percent, from 10 to 20 percent, or from 15 to 25 percent) of the γδ TCR⁺ cells of that population can be BTLA⁺ cells, (n) greater than 60 percent (e.g., greater than 65 percent, greater than 70 percent, greater than 75 percent, greater than 80 percent, greater than 85 percent, greater than 90 percent, or greater than 95 percent) of the γδ TCR⁺ cells of that population can be NKG2D⁺ cells, and (o) greater than 20 percent (e.g., greater than 25 percent, greater than 30 percent, greater than 35 percent, greater than 40 percent, greater than 45 percent, greater than 50 percent, greater than 55 percent, greater than 60 percent, greater than 65 percent, greater than 70 percent, greater than 75 percent, greater than 80 percent, greater than 85 percent, greater than 90 percent, or greater than 95 percent) of the γδ TCR⁺ cells of that population can be NKp46⁺ cells.

In addition to providing the cell populations described herein and the methods for producing those cell populations as described herein, this document provides methods for using the cell populations described herein to treat any appropriate disease, disorder, or condition. For example, the cell populations described herein can be used to treat autoimmune conditions such as rheumatoid arthritis, systemic lupus erythematosus, and scleroderma, infections such as HIV infections, malaria, tuberculosis, hepatitis B, and SARS-CoV-2 infections, and/or cancer. For example, a cell population described herein can be administered to a mammal for use in, for example, adoptive cellular therapies to treat cancer. Any appropriate mammal can be treated with a cell population described herein. For example, humans, horses, cattle, pigs, dogs, cats, mice, and rats can be treated with a population of expanded tumor infiltrating γδ T cells described herein. Any appropriate number of cells can be within the cell population described herein that is administered to a mammal (e.g., a human) to treat cancer. For example, a cell population described herein can have from about 1×10⁷ to about 1×10¹² cells (e.g., from 5×10⁷ to 1×10¹¹ cells, from 1×10⁸ to 1×10¹¹ cells, from 5×10⁸ to 1×10¹¹ cells, from 1×10⁹ to 1×10¹¹ cells, or from 1×10¹⁰ to 1×10¹² cells) and can be administered to a mammal (e.g., a human) to treat cancer. In some cases, a cell population described herein can be administered to a mammal (e.g., a human) to treat cancer such that from about 1×10⁷ to about 1×10¹² (e.g., from 5×10⁷ to 1×10¹¹, from 1×10⁸ to 1×10¹¹, from 5×10⁸ to 1×10¹¹, from 1×10⁹ to 1×10¹¹, or from 1×10¹⁰ to 1×10¹²) of γδ T cells are delivered to the mammal.

Any appropriate cancer can be treated using a cell population described herein. For example, glioblastomas, head & neck squamous cell carcinomas, cutaneous melanomas, lung adenocarcinomas, lung squamous cell carcinomas, breast carcinomas, mesotheliomas, liver hepatocellular carcinomas, pancreatic ductal adenocarcinomas, kidney renal cell carcinomas, bladder urothelial carcinomas, cervical squamous cell carcinomas and endocervical adenocarcinomas, esophageal carcinomas, stomach adenocarcinomas, colorectal adenocarcinomas, rectal adenocarcinomas, ovarian serous cystadenocarcinomas, uterine corpus endometrial carcinomas, prostate adenocarcinomas, and sarcomas can be treated using a cell population described herein.

Any appropriate route of administration can be used to administer a cell population described herein to a mammal. For example, a cell population described herein can be administered intravenously, intraperitoneally, or intratumorally.

When treating a mammal having a condition (e.g., an autoimmune condition), a disease, or an infection (e.g., an HIV infection, malaria, tuberculosis, hepatitis B, and SARS-CoV-2 infection) other than cancer, any appropriate tissue source can be used to obtained γδ T cells. For example, when expanding γδ T cells to treat an autoimmune condition, tissue involved in the autoimmune condition containing γδ T cells or uninvolved tissue containing γδ T cells (e.g., involved or uninvolved skin, liver, kidney, esophagus, small bowel, and/or colon tissue) can be used as a tissue source to obtain γδ T cells as described herein. When expanding γδ T cells to treat an infection, tissue involved in the infection containing γδ T cells or uninvolved tissue containing γδ T cells (e.g., involved or uninvolved skin, liver, kidney, esophagus, small bowel, and/or colon tissue) can be used as a tissue source to obtain γδ T cells as described herein.

The invention will be further described in the following examples, which do not limit the scope of the invention described in the claims.

### EXAMPLES

### Example 1—Expanded Tumor Infiltrating γδ T Cells Demonstrate Potential Utility for Cancer Adoptive Cell Therapy

### Clinical Cohorts and Sample Collection

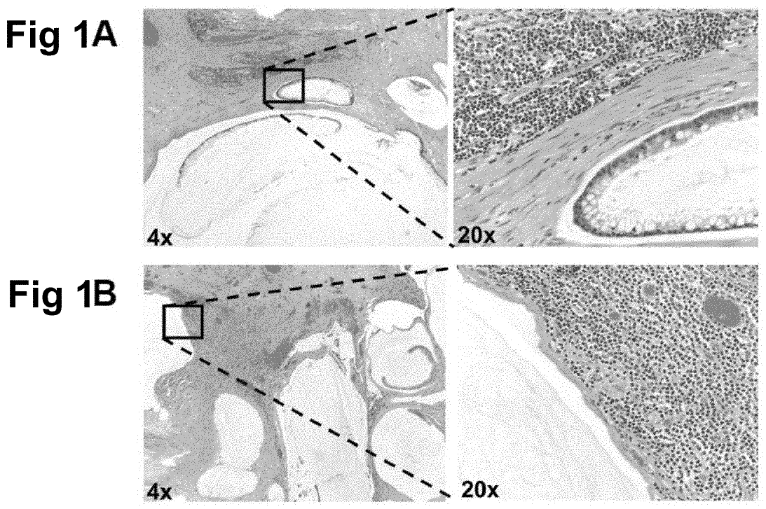

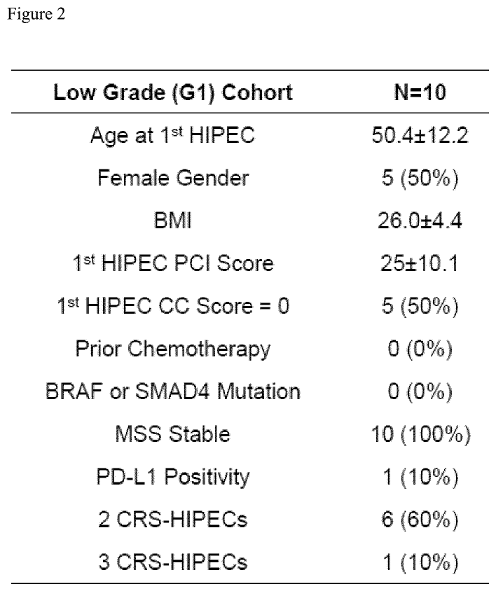

A retrospective series of n=10 low grade (AJCC 8^{th} edition Grade 1) PMP tumors specimens were identified from the University of Pittsburgh Medical Center Digestive Diseases Tissue Repository for immune repertoire sequencing following pathologic review for evidence of lymphocytic infiltrate (FIGS. 1A, 1B, and 2). All selected FFPE tumor specimens were obtained from patients with pathology confirmed grade 1 PMP who underwent CRS-HIPEC with no prior therapies. 5 FFPE tissue scrolls cut at a depth of 6 μm were placed in RNase/DNase free Eppendorf tubes, stored at 4° C. until further processing.

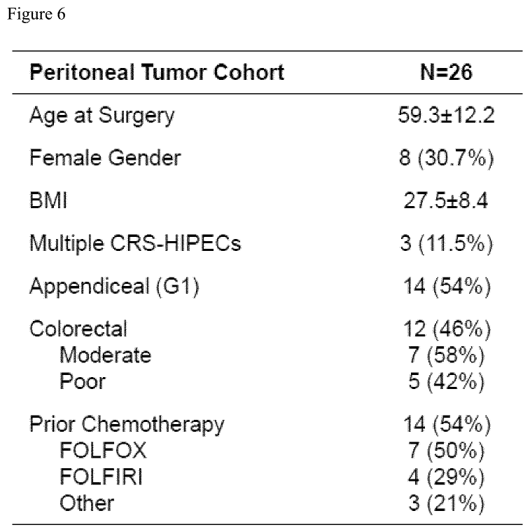

Prospective TIL expansion was completed on n=26 consenting patients with peritoneal surface malignancies (PMP or colon cancer) undergoing standard of care CRS-HIPEC at the University of Pittsburgh Medical Center as part of a non-interventional tumor registry and tissue procurement clinical protocol (FIG. 6). Peritoneal tumor tissue was stored at 4° C. until further processing. Cryopreserved patient buffy coats were retrieved from the UPMC Digestive Diseases Tissue Repository at the time of TIL autologous tumor reactivity testing.

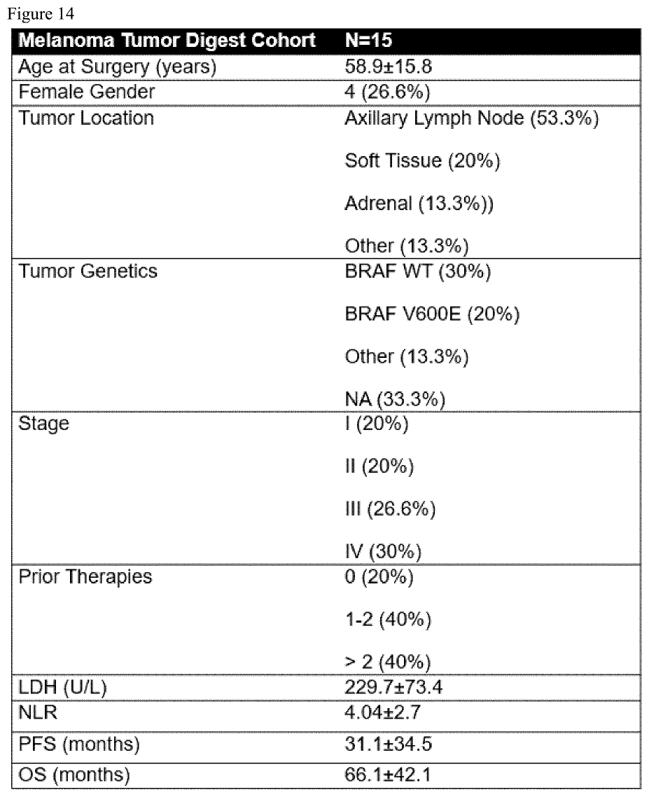

The pre-rapid expansion protocol (pre-REP) modulation of γδ TIL was assessed on n=15 tumor digests from patients undergoing resection for melanoma as part of a non-interventional tumor banking clinical protocol (FIG. 14).

### PMP Immune Repertoire Sequencing and Analysis

Total RNA was extracted from FFPE tissue scrolls using the Covaris M220 focused ultasonicator and truXTRAC FFPE total NA magnetic bead Ultra Kit according to the manufacturer's protocol. The iR-RepSeq-plus 7-Chain DAM-PCR amplification sequence kit (iRepertoire Inc) was used to generate next generation sequencing libraries covering the human TCR-Vα, -Vβ, -Vγ, and -Vδ, and BCR IgH, Igκ, and Igλ chains. 1000 ng of extracted RNA was amplified in a single assay incorporating unique molecular identifiers (UMIs) during the reverse transcription (RT) step by the Biomek-i5 workstation (Beckman Coulter). Amplified libraries were multiplexed and pooled for sequencing on the Illumina NovaSeq platform with a 500-cycle kit. Each sample was allotted 5 million total sequencing reads. Raw data was demultiplexed and UMI guided assembly was performed using migec v1.2.9, and the resulting consensus fastqs were aligned and assembled into clonotypes using mixcr v3.0.14. The output T cell receptor sequence covers FR2 to FR4, as well as the beginning of the constant region.

Raw data were analyzed using the iRmap program (iRepertoire Inc.). Total reads were normalized to generate UMIs, and unique CDR3s (uCDR3s), mean CDR3 length, and sample Shannon true entropy scores were compared across all seven chains. The IgH chain immunoglobulin fraction was assembled with the TRUST algorithm and correlated with TCR and BCR repertoire metrics. Corollary immune repertoire analysis was completed on n=68 pancreatic tumor specimens from patients receiving neoadjuvant chemotherapy and curative resection and n=238 healthy donor PBMCs (iRepertoire). The publicity of PMP TCRs and BCRs was determined by the percent sharing with the n=238 health donor PBMCs. The generational probability of shared PMP specific BCR IgH clonotypes was calculated with the OLGA algorithm.

### TIL Expansion

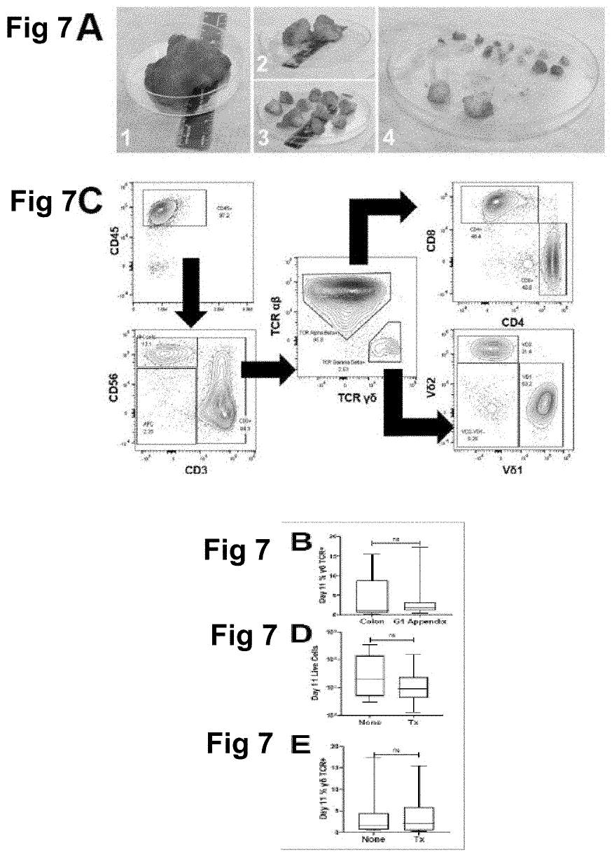

Mucinous peritoneal tumors were dissected to remove necrotic or fatty tissue and n=40 spatially distinct 2-3 mm³ tumor fragments (FIG. 7A) were placed in a gas permeable G-REX™ 100 Flask with complete media (RPMI 1640 (Cytiva HyClone™) supplemented with 10% human AB serum (Gemini Bio), 1% GlutaMAX (Gibco), 5% Penstrep, 1.25 μg/mL Amphotericin B (Gibco), 0.05 μMolar Mercaptoethanol (OME)), and 3,000 IU/mL IL-2 (aldesleukin, Clinigen Therapeutics) as described elsewhere (Jin et al., J Immunother., 35:283-292 (2012)). For experiments expanding TIL from tumor digest, cryopreserved single cell suspensions were thawed, washed twice in PBS, filtered through a 70 μM cell strainer, and counted with a Cellometer K2 Fluorescent Cell Viability Counter (Nexcelom Bioscience). 5×10⁶ cells were plated in a G-REXT™ 6 Well Flask with complete media and respective γ-chain cytokines (IL-4, 100 ng/mL; IL-15, 70 ng/mL; Miltenyi Biotec) or CD137 antibody (Urelumab, 10 μg/mL, Creative Biolabs). G-REX flasks were incubated in a humidified incubator at 37° C. in 5% CO2, and 5 days after culture initiation, half the media was removed and replaced with fresh media and IL-2. After day 5, half the media and IL-2 was replaced every 2 days. On day 11 of culture, TIL were filtered through a 70 μM cell strainer, and counted. 2×10⁶ harvested TIL were saved for spectral cytometry phenotyping and αβ rapid expansion protocol (REP) and the remaining TIL (maximum of 200×10⁶) were used for negative γδ selection with the αβ TCR⁺ magnetic bead isolation kit (Miltenyi Biotec) according to the manufacturer's protocol. A minimum of 0.3×10⁶ αβ TCR⁺ depleted TIL were assessed by spectral cytometry for γδ TIL purity. Paired 1×10⁶ γδ and αβ TIL were expanded under the REP protocol with complete media supplemented with 5% human AB serum and 50% CTS AIMV (Gibco) media, mitogenic OKT-3 (30 ng/mL, Miltenyi Biotec), 1:100 allogenic irradiated feeder cells (2 pooled CMV negative healthy donors, San Diego Blood Bank), IL-2 (3,000 IU/mL) and γ chain cytokines (IL-4, 100 ng/mL; IL-7, 20 ng/mL; IL-15, 70 ng/mL; all Miltenyi Biotec) where indicated. 7 days following the initiation of REP, cultures were counted, resuspended and split 50% into new G-REX flasks and supplemented with fresh CTS AIMV media and cytokines. Cells were counted again on the 10^{th} day of the REP, and half the media was removed and replaced with fresh CTS AIMV and cytokines. On the 14^{th} day of the REP (25^{th} day of culture), TIL were pooled and counted. 1×10⁶ γδ and αβ TIL were saved for spectral cytometry phenotyping, and the remaining expanded TIL were cryopreserved in 10% DMSO in Fetal Bovine Serum with a CoolCell® (Corning) freezing system in −80° C. and transferred to a liquid nitrogen freezer within 24 hours for long term storage.

### Tumor Digestion

Following randomization and selection of n=40 spatially distinct 2-3 mm³ tumor fragments, the remaining tumor fragments (if available) were enzymatically and mechanically digested into a single cell suspension with the human tumor dissociation kit (Miltenyi Biotec) and OctoMACS with Heaters Disassociater (Miltenyi Biotec) according to the manufacturer's protocol. Digested single cell suspensions were filtered with a 70 μm strainer, treated with 10 mL ACK lysis buffer (Gibco) for 5 minutes, washed twice with PBS, counted, and cryopreserved as described above.

### Whole Blood PBMC Isolation

Whole blood was collected in BD Vacutainer® EDTA tubes, diluted 1:1 with PBS and centrifuged atop 15 mL Lymphoprep™ density gradient media (Stemcell Technologies) in a SepMate™ 50 Tube at 1200 G, 20 minutes. Plasma and mononuclear cells were removed, washed with PBS, treated with 10 mL ACK lysis buffer (Gibco) for 5 minutes, washed twice with PBS, counted, and cryopreserved as described above.

### Spectral Cytometry

Day 11 bulk TIL cultures, αβ TCR⁺ depleted cells, and post-REP Day 25 TIL cultures were utilized for spectral cytometry to assess the phenotypic expression of T cell memory, activation, exhaustion, and NCRs. Cells were strained with a 30 μm filter, washed with cytometry buffer (2% FBS in 4° C. PBS), incubated 5 minutes with Human TruStain FcX™ block (Biolegend), washed, and stained with a master mix of fluorescent conjugated antibodies and Brilliant Stain Buffer (BD) for 25 minutes at 4° C. protected from light. Samples were washed and resuspended in 200 μL of cytometry buffer and analyzed on the 5 laser Cytek® Aurora Spectral Cytometer. Single color spectral signatures were measured with UltraComp eBeadsrm (Invitrogen) and spectrally resolved along with TIL autofluorescence spectral signature using the SpectroFlo® software. Following gating of single cell, live, CD45⁺ immune cell, CD56⁺ NK cells, CD3⁺ cells, αβ TCR⁺ CD4⁺ and CD8⁺ T cells, and γδ TCR⁺Vδ1⁺, Vδ2⁺, and Vδ1⁻Vδ2⁻ T cells (FIG. 7C), expression of phenotypic markers was assessed based on fluoresce minus one (FMO) controls on FlowJo v10.7 software. Expression of phenotypic markers within αβ and γδ TCR⁺ TIL was assessed on day 11 and day 25 of culture, and the change in the expression of the markers between the two time points was assessed within αβ and γδ TCR⁺ TIL populations.

### TIL-Tumor Reactivity

To assess the autologous tumor reactivity of expanded αβ (IL-2 only) and γδ (IL-2, IL-4, and IL-15) TIL, cryopreserved TIL were thawed and rested overnight in IL-2 (3,000 IU/mL) media, washed twice with PBS, counted and plated (1×10⁵ cells) in a 96 well round bottom plate in IL-2 free complete media alone, with CD3-CD28 stimulation (Dynabeads, 2.5 μL/well, Invitrogen), 1×10⁵ autologous PBMC, or 1×10⁵ autologous tumor digest single cell suspension with culture volume normalized to 200 μL for 24 hours in a humidified incubator at 37° C. in 5% CO2 as described elsewhere (Dudley et al., J. Immunother., 26:332-342 (2003)). 50 μL of supernatant was harvested from duplicate co-cultures, diluted 1:2, and assessed for production of IFNγ with the Human IFNγ ELISA Kit (Invitrogen) according to the manufacturer's instructions. TIL-autologous tumor digest reactivity was compared with co-culture with autologous PBMC and between paired γδ and αβ TIL. In certain cases, γδ TIL or autologous tumor digest were also incubated with blocking antibodies (TIL: isotype control mouse IgG (Invitrogen, 10 μg/mL), anti-γδ TCR (Novus Biologicals, clone 7A5, 3 μg/mL), or anti-NKG2D (BD, clone 1D11, 10 μg/mL) or tumor digest: isotype control mouse IgG (10 μg/mL) or anti-MHC-1 (Invitrogen, W6/32 10 μg/mL) for 2 hours prior to co-culture.

To assess the MHC unrestricted recognition of TIL, γδ and αβ TIL were similarly co-cultured with 1×10⁵ cancer cell lines (K562, HCT116, RKO, SW480, or SW48; all from ATCC, authenticated and Mycoplasma negative (eMyco™ plus PCR kit)) following a minimum of two passages of culture in complete media in a humidified incubator at 37° C. in 5% CO2.

### Cancer Cell Encyclopedia (CCLE) Analysis

To evaluate the expression of NKG2D ligands in the tested cancer cell lines, mRNA Z-scores of MICA, MICA, ULBP1, ULBP2, and ULBP3 were queried for the K562, HCT116, RKO, SW480, and SW48 cells from the Cancer Cell Line Encyclopedia using the cBioPortal for cancer genomics.

### TCGA Analysis

The tumor specific Vδ1 infiltration and prognostic ability was assessed in the 20 most common primary solid tumors (NCI) of bulk RNA sequencing data in The Cancer Genome Atlas (TCGA) with the Gene Expression Profiling Interactive Analysis Server 2 (GEPIA2). Mean expression (log transcripts per million) of γδ TIL subsets (TRDV1, TRDV2, and TRDV3) and αβ TIL (TRBC2 Beta Chain 2 Constant Region) were calculated. Kaplan Meier survival analysis by normalized (ACTB beta actin) TRDV1 expression above (high) or below (low) the median for selected tumor types was completed with calculation of Log rank p value and 95% confidence interval of survival estimates. TRDV1 expression was directly correlated with TRBC2 expression across selected tumors, and corresponding Pearson correlation coefficient and P values were calculated.

### Statistical Analysis

Data were expressed as mean±standard deviation. Graphical visualization and statistical analysis were performed using Microsoft Excel and GraphPad Prism 9. Descriptive statistics, Two-tailed non-parametric test, Mann-Whitney U tests (unpaired), and Wilcoxon signed-rank (paired, for all comparisons of αβ and γδ TIL) tests were used. Correlations were calculated with the Pearson correlation coefficient and plotted with nonlinear regression and 95% confidence bands. P values<0.05 were considered statistically significant, and significance levels were set to * P<0.05, ** P<0.01, *** P<0.001, and **** P<0.0001.

### Results

Low Grade Pseudomyxoma Peritonei (PMP) Display Elevated B Cell Receptor (BCR) IgE Fraction Associated with TCR Vδ

Following pathologic analysis of previously resected peritoneal tumor specimens (FIGS. 1A and 1B), a representative cohort (n=10) of treatment naïve patients with low grade (GI) PMP who were treated with standard of care cytoreductive surgery and heated 10 intraperitoneal chemotherapy (CRS-HIPEC, FIG. 2) were identified. All patients displayed microsatellite stable tumors, limited programmed death ligand-1 (PD-L1) positivity (n=1), with 7 patients requiring at least one follow-up CRS-HIPEC for tumor recurrence. H&E staining identified infiltrating lymphocyte populations restricted to the tumor associated stroma that were notably absent from mucin pools (FIGS. 1A and 1B).

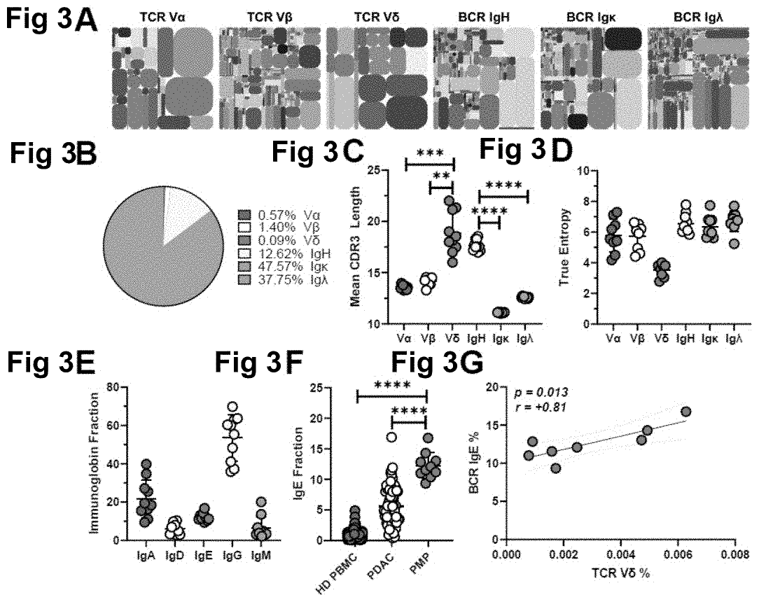

With limited prior understanding of the adaptive immune response to this understudied tumor type, full TCR and BCR sequencing of the resected FFPE tumor specimen from the first operative resection (FIGS. 3A-3G) was completed. Following dimer avoidance multiplex PCR (DAM-PCR) of the bulk tumor RNA, cDNA library preparation, and NGS, complete unique CDR3 (uCDR3) sequences were constructed with the migec v1.2.9 MixCR pipeline for adaptive immune repertoire analysis (Han et al., Cancer Treat. Res., 180:111-147 (2020); Han et al., Methods Mol. Biol., 2055:369-397 (2020); and Bolotin et al., Nat. Methods, 12:380-381 (2015)). Representative tree maps of a single patient's TCR Vα, VD, Vδ, and BCR IgH, Igκ, Igλ repertoire are shown in FIG. 3A. Notably, the seven-chain repertoire was predominantly made up of BCR transcripts with only 2.06% of total average reads accounting for TCR clones that were primarily derived from αβ T cells (FIG. 3B). With the Vδ chain only being 0.09% of reads on average, Vγ reads were predictably not verifiably detected. As previously reported, the CDR3 length of the Vδ chain (18.8±2.0 amino acids) was greater and more variable than the Vu (13.5±0.2, p<0.001) and VP (14.1±0.3, p=0.005) chains (FIG. 3C) (Rock et al., J. Exp. Med., 179:323-328 (1994)). The Vδ chain was similar in length to IgH (17.6±0.5), which was greater than the Igκ (11.1±0.03, p<0.0001) and Igλ (12.5±0.09, p<0.0001) chains. Calculation of the true Shannon entropy of the repertoires exhibited similar diversity across chains, although the Vδ was markedly decreased compared to Vαβ and BCR repertoires (FIG. 3D) (Bortone et al., Cancer Immunol. Res., 9:103-112 (2021)).

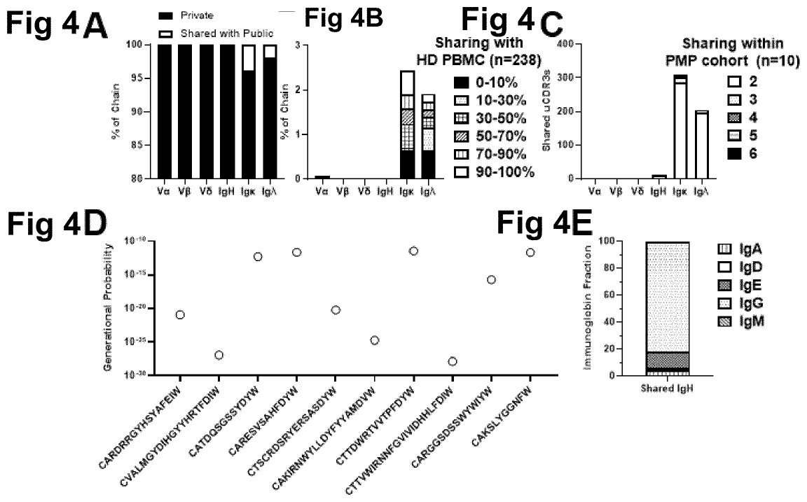

Comparison with a cohort of healthy donor PBMC repertoires (n=238) revealed that this low grade PMP cohort displayed a highly private repertoire with only Vu (0.06% of chain), Igκ (3.7%), and Igλ (1.9%), demonstrating shared public CDR3s (FIGS. 4A-B). Analysis of shared CDR3s within the patient group identified shared putative convergent disease specific BCR, but not TCR CDR3s (FIG. 4C). Given that shared disease associated CDR3s can arise from both random recombination and convergent evolution of antigen driven recombination, the generational probability of these shared BCR CDR3s were calculated, revealing a spectrum of antigen driven IgH CDR3s, that were primarily IgG or IgE (FIGS. 4D-E) (Murugan et al., Proc. Natl. Acad. Sci. USA, 109:16161-16166 (2012); and Sethna et al., Bioinformatics, 35:2974-2981 (2019)).

Given the unexpected abundance of BCR transcripts, the immunoglobin fraction of the total low grade PMP IgH repertoire was further analyzed, which revealed an expected distribution dominated by IgG (53.4±12.0%) and IgA (21.7±9.9%) (FIG. 3E). However, an unusually elevated IgE fraction (12.2±2.1%) that was substantially greater than that observed in healthy donor PBMC (n=238, 0.9±0.6%, p<0.0001) and high-grade pancreatic cancer tumor (n=68, 5.6±3.2%, p<0.0001) repertoires was observed (FIG. 3F). When correlating the BCR IgE fraction with other repertoire features, a strong positive correlation with Vδ expression (r=+0.81, p=0.013), but not with Vu or VO chains, was observed (FIG. 3G). The IgE expression levels and association with γδ TIL was intriguing as intraepithelial γδ T cells were previously shown to be required for tumor protective IgE class switching in response to epithelial DNA damage (Crawford et al., Nat. Immunol., 19:859-870 (2018)).

### γδ TIL Sparsely Infiltrate Peritoneal Surface Malignancies

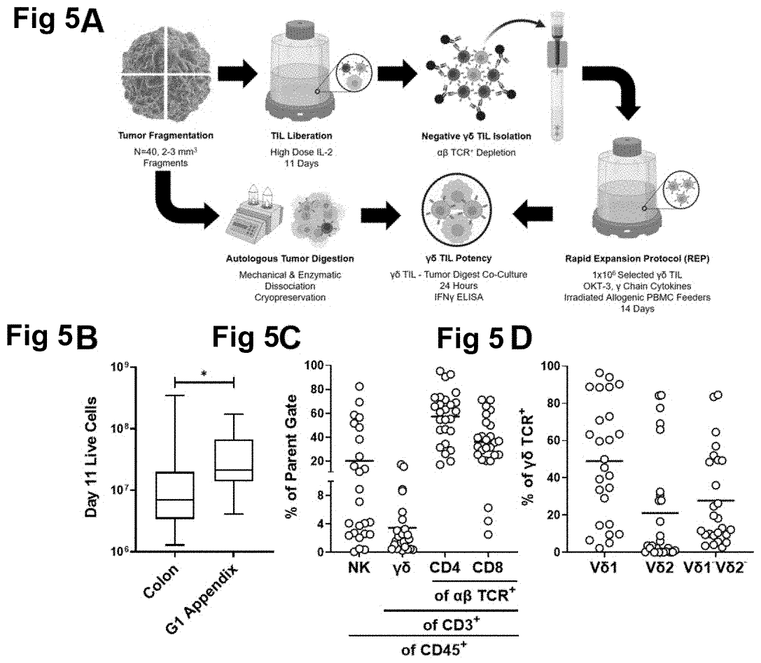

With the understanding that peritoneal γδ TIL display a diverse polyclonal and private repertoire, the following was performed to prospectively assess γδ TIL. Tumor specimens were collected from consenting patients with peritoneal surface malignancies undergoing CRS-HIPEC (n=26) (FIG. 5A). 30.7% of patients were female with an average of age of 59.3±12.2 and BMI of 27.5±8.4 (FIG. 6). Patients in this prospective cohort had peritoneal tumors of low grade appendiceal (n=14, 54%) and high grade colorectal (n=12, 46%) cancer. Three patients (11.5%) previously underwent CRS-HIPEC with 14 patients (54%) previously receiving systemic chemotherapy.

Mucinous peritoneal tumors were dissected into spatially distinct 2-3 mm³ fragments (FIG. 7A) and cultured in gas permeable rapid expansion flasks (G-REX™). Peritoneal TIL were liberated with high dose IL-2 (3,000 IU/mL) and harvested following 11 days of culture. While there was no difference in the number of total viable TIL between treatment naïve patients and those receiving preoperative chemotherapy (FIG. 7B), low grade appendix cancers (4.9×10⁷±6.1×10⁷ cells) had greater total viable TIL compared to high grade colon cancers (3.9×10⁷±10.3×10⁷ cells, p=0.028, FIG. 5B).

Multispectral flow cytometry was utilized to define the composition and phenotype of the bulk proliferating peritoneal TIL populations. IL-2 reactive CD56⁺ CD3⁻ Natural Killer (NK) cells and CD3⁺ T cells were the major constituents of the CD45⁺ TIL population (FIG. 5C). NK cells represented on average 20.2% of all CD45⁺ cells, with certain individual patients having less than 4% and others having greater than 40% NK cells (FIG. 5C). γδ TCR⁺ cells (3.4±4.4%) represented a small fraction of all CD3⁺ T cells, which were primarily CD4⁺ (57.3±21.7) or CD8⁺ (36.1±19.3) αβ TCR⁺ cells. CD3⁺ γδ TCR⁺ cells were primarily Vδ1⁺ (49.0±31.5%) or VS1⁻Vδ2⁻ (27.7±25.1%), with Vδ2⁺ (21.0±29.5%) cells being less prevalent on average, despite accounting for greater than 60% of γδ TCR⁺ cells in five patients (FIG. 5D). Despite considerable heterogeneity of γδ TIL populations, no substantive differences in γδ TCR⁺ cell phenotypes were observed between patients with appendiceal or colon tumors or with prior treatment (FIGS. 7D-E).

γδ TIL Display a Tissue Resident Effector Memory Phenotype with Reduced PD-1, but Greater NKG2D and CD137 Expression Compared to αβ TIL

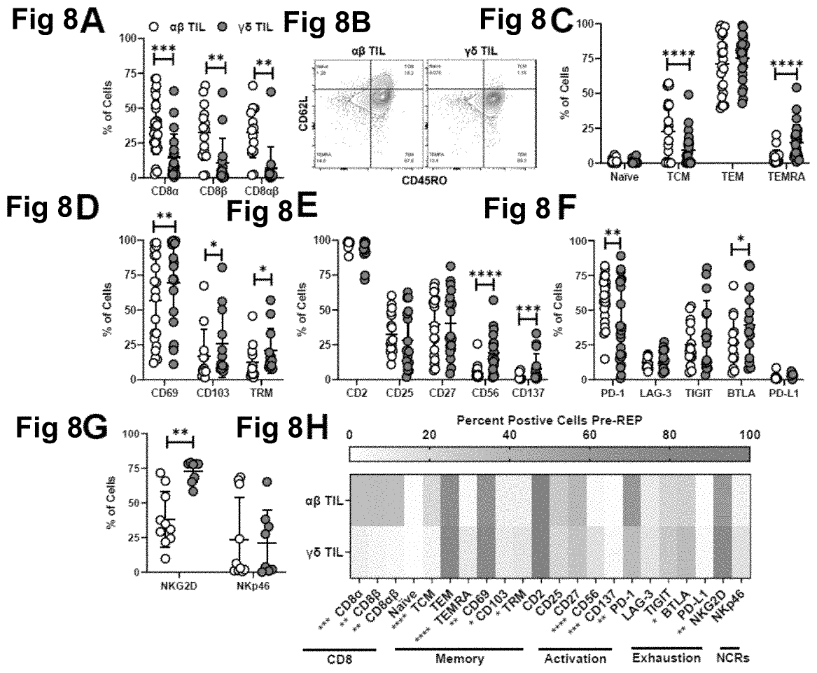

To better understand the phenotype of the γδ and up TIL populations, markers of T cell memory, differentiation, and activation, inhibitory receptors associated with T cell exhaustion, and expression of natural cytotoxicity receptors (NCRs) were assessed (FIGS. 8A-H and 9A-E). Compared to CD8-γδ T cells, intraepithelial CD8αβ⁺γδ T cells typically display a heightened T helper type 1 (Th1) phenotype associated with gut homeostasis and mucosal healing (Mikulak et al., JCI Insight, 4(24):e125884 (2019); and Kadivar et al., J. Immunol., 197:4584-4592 (2016)). In this subset of expanded peritoneal TIL, CD8α⁺ (14.6±36.1%), CD8β⁺ (11.0±17.4%), or CD8αβ⁺ (6.69±15.3%) γδ TIL represented a small fraction of cells, much lower than corresponding αβ TIL in individual cultures (FIG. 8A).

The majority of γδ TIL displayed an effector memory phenotype (TEM: CD45RO⁺ CD62L⁻, 75.5±15.8%) that was comparable to that observed in αβ TIL (71.2±20.8%, FIGS. 8B-C). At 11 days of culture following initial resection, αβ TIL had a greater proportion of central memory cells (T_{CM}: CD45RO⁺ CD62L⁺, 22.4±21.7% vs 9.1±12.0%, p<0.0001) compared to γδ TIL. γδ TIL also had a relative greater proportion of terminally differentiated effector memory RA cells (T_{EMRA}: CD45RO⁻, CD62L⁻, 14.9±12.9% vs 4.4±4.6%, p<0.0001). Tissue resident memory T cells (T_{RM}) expressing the tissue retention markers CD69 and CD103 display long term protective immunity and are associated with improved outcomes following immunotherapy (Okla et al., J. Exp. Med., 218(4):e20201605 (2021)). γδ TIL displayed higher amounts of CD69⁺ (69.9±30.5% vs 56.6±31.8%, p=0.003, FIG. 8D), CD103⁺ (25.8±24.1% vs 16.6±19.4%, p=0.016), and double positive T_{RM} cells (20.8±16.2 vs 12.3±13.0, p=0.020) compared to αβ TIL.

Given that the composition of ex vivo expanded TIL populations is highly dependent on spatial heterogeneity and culture conditions promoting the proliferation of tumor dominant and minority populations associated with differential tumor reactivity, expression of activation and exhaustion molecules (Poschke et al., Clin. Cancer Res., 26:4289-4301 (2020)) were compared. Expanded γδ (92.2%) and αβ (97.4%) TIL displayed high levels of CD2 (FIG. 8E), a costimulatory molecule whose signaling enables immunologic synapse formation, the so-called CD2 corolla, and buffers PD-1 mediated exhaustion (McKinney et al., Nature, 523:612-616 (2015); and Demetriou et al., Nat. Immunol., 21:1232-1243 (2020)). The IL-2 receptor a chain (CD25) was moderately expressed on γδ (28%) and αβ (32.4%) TIL. The costimulatory tumor necrosis receptor family member CD27 has been implicated as a thymic regulator of interferon γ (IFNγ) expression over IL-17 producing γδ T cells (Ribot et al., Nat. Immunol., 10:427-436 (2009); and Ribot et al., Cell. Mol. Life Sci., 68:2345-2355 (2011)). Increased CD27⁺ T cells have also been associated with objective clinical response in a prior trial of predominantly αβ TIL therapy (Rosenberg et al., Clin. Cancer Res., 17:4550-4557 (2011)). γδ TIL showed a range of expression of CD27 that on average (40.2%) was similar to that of αβ (39.5%) TIL. Besides identifying NK cells, the neural cell adhesion molecule, CD56, is a marker of enhanced T cells Th1 cytokine production and cytolytic capability and was expressed to a substantially greater degree in γδ (19.2±14.1%) than αβ TIL (4.5±5.4%, p<0.0001) (Kelly-Rogers et al., Hum. Immunol., 67:863-873 (2006); Cohavy et al., J. Immunol., 178:5524-5532 (2007); and Almehmadi et al., Immunology, 142:258-268 (2014)). Upregulation of CD137 (4-1BB) has been identified as a marker of tumor reactive T cells with enhanced clonal expansion and proliferation (Cooper et al., Eur. J Immunol., 32:521-529 (2002); and Ye et al., Clin. Cancer Res., 20:44-55 (2014)). CD137 expression under these conditions was low across all cells, but notably higher on γδ (8.0±10.5%) when compared to αβ (1.8±2.3%, p=0.0002) TIL.

Inhibitory immune receptor expression are simultaneous markers of tumor reactivity, immune exhaustion, and potential for suppression (Ahmadzadeh et al., Blood, 114:1537-1544 (2009); Baitsch et al., J Clin. Invest., 121:2350-2360 (2011); Miller et al., Nat. Immunol., 20:326-336 (2019); and Gros et al., J Clin. Invest., 124:2246-2259 (2014)). With the exception of PD-L1, γδ TIL displayed more variable expression of PD-1, LAG-3, TIGIT, and BTLA compared to αβ TIL (FIG. 8F). PD-1 was lower on γδ (39.4±27.4%) compared to αβ (57.7±16.9%, p=0.004) TIL. Expression of LAG3 (12.2% and 14.8%) and TIGIT (25.2% and 31.5%) were generally expressed at lower levels than PD-1 for both αβ and γδ TIL subsets. BTLA, a dual regulator of T cell co-stimulation and suppression of TCR signaling, is a marker of enhanced T cell survival and TIL therapy response that exhibited somewhat higher expression on γδ (39.5±25.3%) compared to αβ (26.6±18.0%, p=0.032) TIL (Radvanyi et al., Clin. Cancer Res., 18:6758-6770 (2012); Haymaker et al., Oncoimmunology, 4:e1014246 (2015); and Ritthipichai et al., Clin. Cancer Res., 23:6151-6164 (2017)). In addition to being expressed on tumor cells, suppressive myeloid populations, and T regulatory cells, PD-L1 expression on effector T cells promotes self-tolerance and accelerated tumorigenesis in murine models (Daley et al., Cell, 166:1485-1499 e1415 (2016); and Diskin et al., Nat. Immunol., 21:442-454 (2020)). PD-L1 expression was low for both expanded γδ (3.4%) and up (1.8%) TIL.

The innate-like NK cell properties of γδ T cells, including expression of the NCRs NKG2D and NKp46 confer additional reactivity to stress antigens and antitumor potential (Silva-Santos et al., Nat. Rev. Cancer, 19:392-404 (2019); Wu et al., Sci. Transl. Med., 11(513):aax9364 (2019); Mikulak et al., JCI Insight, 4(24):e125884 (2019); and Foord et al., Sci. Transl. Med., 13(577):abb0192 (2021)). While expression of NKG2D was uniformly high on γδ TIL (72.8±7.9%) and higher than αβ TIL (38.0±19.8%, p=0.007), NKp46 expression was more heterogenous (17.4±22.4%) and did not differ from αβ TIL (23.6±30.1%) (FIG. 8G). A summary heatmap of the mean expression of all evaluated phenotypic markers on γδ and αβ TIL are included in FIG. 8H.

### Expansion of γδ TIL

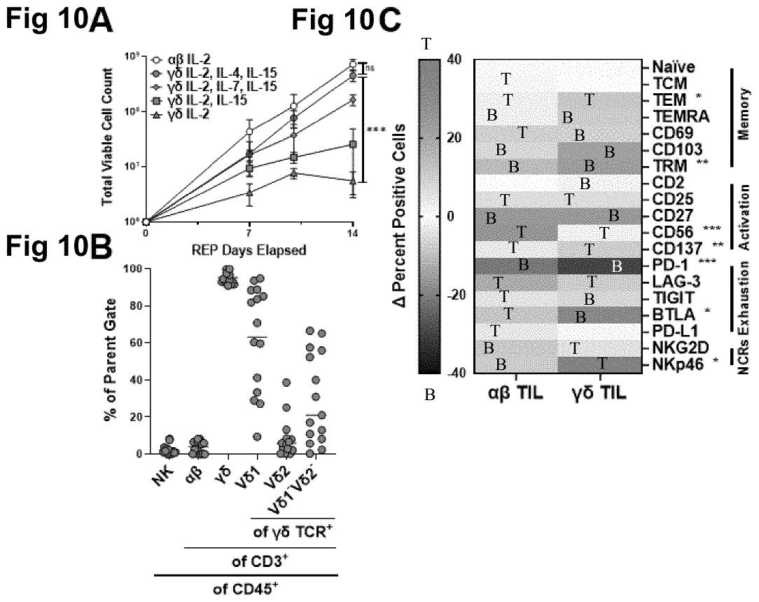

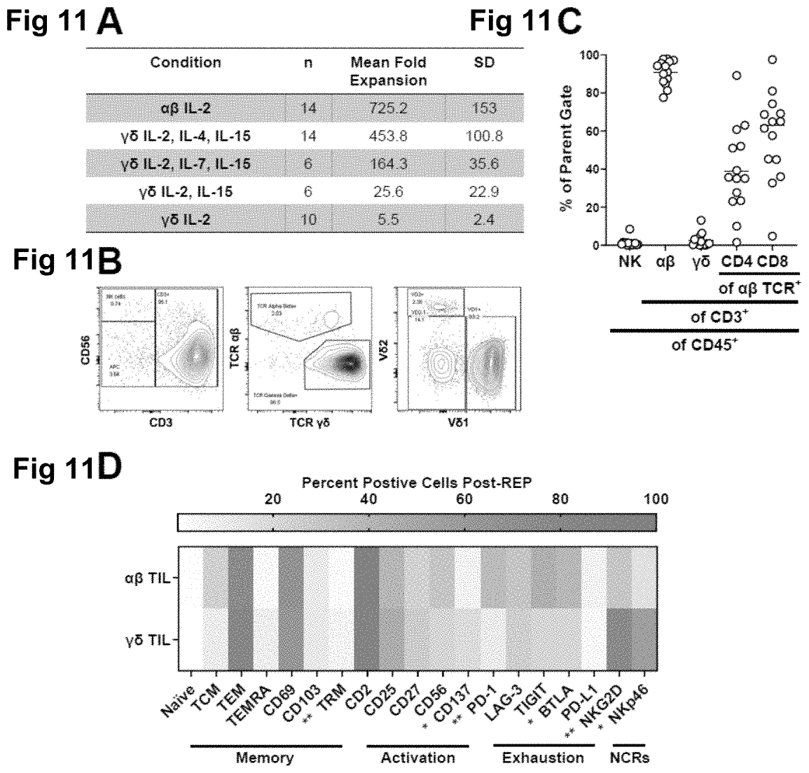

To consider the adoptive transfer of γδ TIL displaying a favorable tissue resident effector memory phenotype with limited exhaustion and enhanced expression of CD137 and NKG2D, an expansion protocol was designed to generate a clinically feasible number of γδ TIL. γδ TIL were negatively selected with depletion of αβ TCR⁺ cells. Then, 1×10⁶ γδ TIL (or bulk αβ TIL for comparison) were expanded for 14 days with mitogenic CD3 stimulation (OKT-3, 30 ng/mL), high concentrations of IL-2 (3,000 IU/mL), and irradiated allogenic healthy donor PBMCs (FIG. 10A). This IL-2-dependent expansion protocol was insufficient to expand γδ TIL (5.5 fold expansion, FIG. 11A) and may explain the limited number of γδ TIL observed in prior TIL therapies (Donia et al., Oncoimmunology, 1:1297-1304 (2012)).

Different combinations of cytokines were evaluated (in combination with anti-CD3 and irradiated PBMCs) to determine if a population of γδ TIL having a desired phenotype can be obtained in appropriate numbers and percentages. While addition of IL-15 (25.6 fold expansion) or IL-7 (164.3 fold expansion) increased expansion of selected γδ TIL, a combination of IL-2, IL-4, and IL-15 (453.8±100.8 fold expansion) demonstrated considerably enhanced γδ TIL expansion (p=0.0008) that was largely comparable to that observed for the IL-2 only expansion of native αβ TIL (725.5±153 fold expansion) (FIG. 11A).

Spectral cytometric phenotyping of the negatively selected γδ TIL that were IL-2/IL-4/IL-15 expanded (FIG. 11B) displayed a high purity of γδ TCR⁺ cells (95.3±3.1% of CD3⁺ cells, FIG. 10B) with minimal NK cells (2.3±2.5% of CD45⁺ cells) or αβ TCR⁺ cells (3.87±3.3% of CD3⁺ cells). The negatively selected γδ TIL that were IL-2/IL-4/IL-15 expanded were predominantly Vδ1⁺ (63.2±28.3% of γδ TCR⁺) or VDδ1⁻Vδ2⁻ (29.8±24.2%) cells with a minor proportion of Vδ2⁺ cells (8.5±10.4%) (FIG. 10B). In comparison, the native αβ TIL that were IL-2 expanded were primarily αβ TCR⁺ (90.8%±6.5% of CD3⁺ cells; which were CD8⁺ (57.3±23.1%) or CD4⁺ cells (39.0±22.8%)), with minimal NK (1.27±2.1%) or γδ TCR⁺ (2.5±3.5%) cells (FIG. 11C).

The IL-2/IL-4/IL-15 expansion of the negatively selected γδ TIL resulted in increased proliferation of TEM γδ TIL (87.1±7.2% vs 75.5±15.8%, p=0.034), with reduced T_{EMRA} (7.0±6.3% vs 14.9±12.9%, p=0.031) compared to the negatively selected γδ TIL preparation before IL-2/IL-4/IL-15 expansion (FIG. 10C). An increased number of infused TEM and reduced number of T_{EMRA} populations are associated with clinical response to TIL therapy (Goff et al., J Clin. Oncol., 34:2389-2397 (2016)).

Following IL-2/IL-4/IL-15 expansion of negatively selected γδ TIL (5.3±2.7% vs 20.8±16.2%, p<0.0001) and IL-2 only expansion of native αβ TIL (1.7±1.5% vs 12.3 13.0%, p=0.004), the number of CD69⁺ CD103⁺ T_{RM} cells were reduced compared to the pre-expansion TIL, but higher in the γδ TIL population (p=0.004, FIG. 11D). Expression of CD2, CD25, and CD27 were generally stable following both IL-2/IL-4/IL-15 expansion of negatively selected γδ TIL and IL-2 only expansion of native up TIL. CD56 expression was increased in the IL-2 only expanded, native αβ TIL (30.3±23.3% vs 4.5±5.3%, p=0.0007), but not increased in the IL-2/IL-4/IL-15 expanded, negatively selected γδ TIL (21.6±25.8% vs 19.2±14.1%) following the expansion as both populations exhibited similar levels of expression. CD137 exhibited increased expression in the IL-2/IL-4/IL-15 expanded, negatively selected γδ TIL following expansion (18.2±13.7% vs 8.0±10.5%, p=0.006) and that level remained higher than the level observed in the IL-2 only expanded, native up TIL (6.18±8.9%, p=0.036). PD-1 expression was reduced in both the IL-2 only expanded, native αβ TIL (36.2±22.5% vs 57.7±16.9%, p=0.030) and the IL-2/IL-4/IL-15 expanded, negatively selected γδ TIL (9.7±7.3% vs 39.4±27.4%, p=0.0006) as compared to pre-expansion, but the level remained lower in the post-expansion γδ TIL compared to the post-expansion αβ TIL (p=0.002). While expression of LAG3 and TIGIT was stable for both populations following expansion, BTLA expression slightly increased in the IL-2 only expanded, native αβ TIL (38.7%±14.1% vs 26.6±18.0%, p=0.129) and slightly decreased in the IL-2/IL-4/IL-15 expanded, negatively selected γδ TIL (20.8±9.8% vs 39.5±25.3%, p=0.154) and was higher in the post-expansion αβ TIL compared to the post-expansion γδ TIL (p=0.030). While αβ TIL did not exhibit altered NCR expression of NKG2D and NKp46 following the IL-2 only expansion, the IL-2/IL-4/IL-15 expanded, negatively selected γδ TIL maintained high expression of NKG2D (77.9±14.2%) and had an increased number of NKp46⁺ expressing cells (56.1±32.8% vs 17.4±22.4%, p=0.011; post-expansion compared to pre-expansion) that was greater than that observed in the IL-2 only expanded, native αβ TIL (15.7±22.3%, p=0.029).

### MHC Independent, γδ TCR Mediated Autologous Tumor Recognition

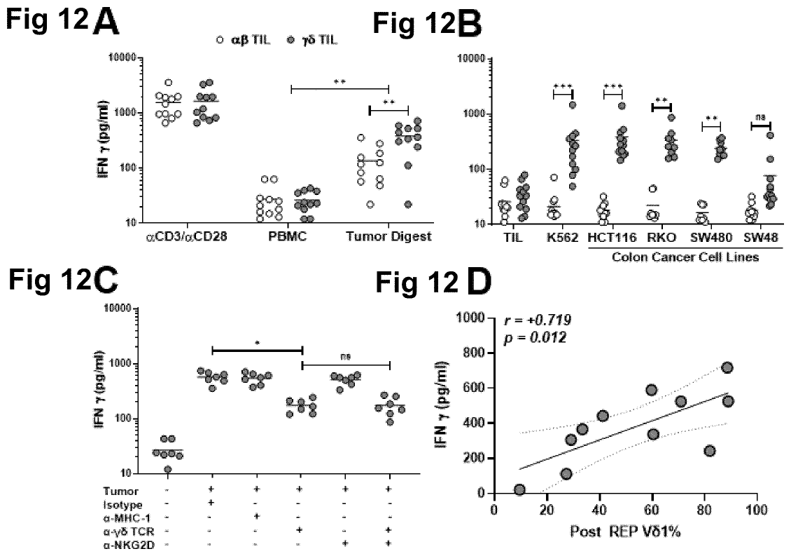

Completed and ongoing trials of TIL therapy in patients with metastatic epithelial cancer have identified in vitro TIL reactivity to autologous patient tumor as a key determinant of objective clinical response (Tran et al., Science, 344:641-645 (2014); Stevanovic et al., J. Clin. Oncol. 33:1543-1550 (2015); Stevanovic et al., Clin. Cancer Res., 25:1486-1493 (2019); Chandran et al., Lancet Oncol., 18:792-802 (2017); and Zacharakis et al., Nat. Med., 24:724-730 (2018)). To measure the tumor reactivity of the expanded peritoneal TIL, in patients with available specimens (n=11), IFNγ production was assessed following 24-hour co-culture of a 1:1 ratio of autologous tumor digest cryopreserved at the time of resection and either IL-2/IL-4/IL-15 expanded, negatively selected γδ TIL or IL-2 only expanded, native αβ TIL (FIG. 12A). Following non-specific stimulation with beads coated with anti-CD3/anti-CD28 mAbs, both αβ (1556±849 μg/mL) and γδ (1638±1023 μg/mL) TIL produced similar levels of IFNγ. Both αβ (135.8±103.5 vs 27.4±18.4 μg/mL, p=0.002) and γδ TIL (380.7±207.6 vs 25.2±12.1 μg/mL, p=0.001) produced significantly greater amounts of IFNγ during co-culture with autologous tumor digest compared TIL co-cultured with autologous PBMC. The γδ TIL displayed greater autologous tumor reactivity when compared with paired αβ TIL (p=0.009). Notably, 6 of 11 (55%) αβ and 10 of 11 (91%) γδ TIL populations produced greater than 100 μg/mL of IFNγ following co-culture with tumor digest, a hypothesized threshold for screening TIL reactivity associated with clinical tumor regression (Chandran et al., Lancet Oncol., 18:792-802 (2017)).

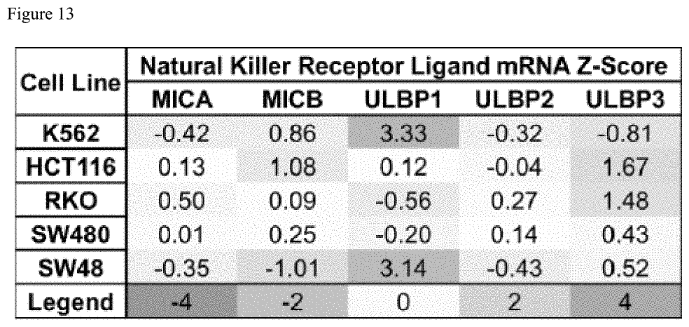

Given that γδ TIL possess MHC unrestricted TCRs, their reactivity against a series of HLA unmatched cancer cell lines also was evaluated (FIG. 12B). Compared to αβ TIL incapable of recognizing such unmatched cell lines, γδ TIL produced significantly higher amounts of IFNγ when cultured with the K562 leukemia cell line and a series of colon cancer cell lines (HCT116, RKO, and SW480). The reactivity of γδ TIL against the SW48 colon cancer cell line was markedly lower than against other cancer cell lines and no different than that observed from the αβ TIL/SW48 co-culture. The γδ TIL's lack of reactivity towards the SW48 line was hypothesizedto be caused by reduced production or expression of γδ TCR or NKG2D antigens. Analysis of the mRNA expression of known ligands of the NKG2D receptor in the evaluated cell lines within the Cancer Cell Encyclopedia (CCLE) identified stable or increased expression of MICA and MICB in K562, HCT116, RKO, and SW480, but reduced expression of MICA (−0.35 Z score) and MICB (−1.01 Z score) in the SW48 line (FIG. 13) (Barretina et al., Nature, 483:603-607 (2012)).

Given the established role of γδ T cell NCR mediated recognition of target cells and uniformly high expression of NKG2D within this cohort of expanded peritoneal γδ TIL, the following was performed to identify its role, along with the γδ TCR, in mediating autologous tumor reactivity (Silva-Santos et al., Nat. Rev. Immunol., 15:683-691 (2015); and Silva-Santos et al., Nat. Rev. Cancer, 19:392-404 (2019)). Following co-culture of IL-2/IL-4/IL-15 expanded, negatively selected γδ TIL with autologous tumor digests (n=7), combinations of anti-MHC-1 (W6/32), anti-NKG2D (1D11), anti-γδ TCR (7A5), or isotype control (mouse IgG) mAb were utilized to block the corresponding receptor binding and signaling (FIG. 12C). While addition of anti-MHC-1 mAb showed no difference in γδ TIL IFNγ production and confirmed MHC independent recognition, addition of anti-γδ TCR mAb significantly reduced IFNγ production compared to blocking with the isotype control. Addition of the 5 anti-NKG2D antibody showed minimal effect on IFNγ production and was not further reduced when blocked in combination with the γδ TCR, suggesting the involvement of the γδ TCR in mediating autologous tumor reactivity.

To identify additional factors associated with γδ TIL autologous tumor reactivity, the production of IFNγ following autologous tumor digest co-culture with IL-2/IL-4/IL-15 expanded, negatively selected γδ TIL phenotypic characteristics were compared. The percent composition of Vδ1 positively correlated (r=+0.719, p=0.012) with IFNγ production, supporting earlier reports of the enhanced anti-tumor potential of Vδ1 cells over that observed with other γδ subsets (FIG. 12D) (Deniger et al., Clin. Cancer Res., 20:5708-5719 (2014); Fisher et al., Clin. Cancer Res., 20:5720-5732 (2014); and Cordova et al., PLoS One, 7:e49878 (2012)).

Pre-Rapid Expansion Protocol Modulation of γ Chain Cytokines and CD137 Engagement does not Improve γδ TIL Expansion

Given the enhanced autologous tumor reactivity of γδ TIL in comparison to αβ TIL, methods for the specific expansion of γδ TIL during the pre-REP culture period, which determines the input number of γδ TIL available for REP, were investigated. With the increased number of γδ TIL following isolation and culture with IL-2, IL-4, and IL-15 during the REP, this γ chain combination was evaluated in a retrospective cohort of cryopreserved tumor digests (n=15, FIG. 14) obtained from consenting patients undergoing resection following initial diagnosis or neoadjuvant therapy for melanoma. In addition to the γ chain combination, a humanized agonistic monoclonal antibody targeting the CD137 receptor (Urelumab, 10 μg/mL) was evaluated given the higher expression of CD137 on γδ TIL and prior reports of enhanced TIL expansion with CD137 engagement (Hall et al., J. Immunother. Cancer, 4:61 (2016); Sakellariou-Thompson et al., Clin. Cancer Res., 23:7263-7275 (2017); Poch et al., Oncoimmunology, 7:e1476816 (2018); Tavera et al., J. Immunother., 41:399-405 (2018)).

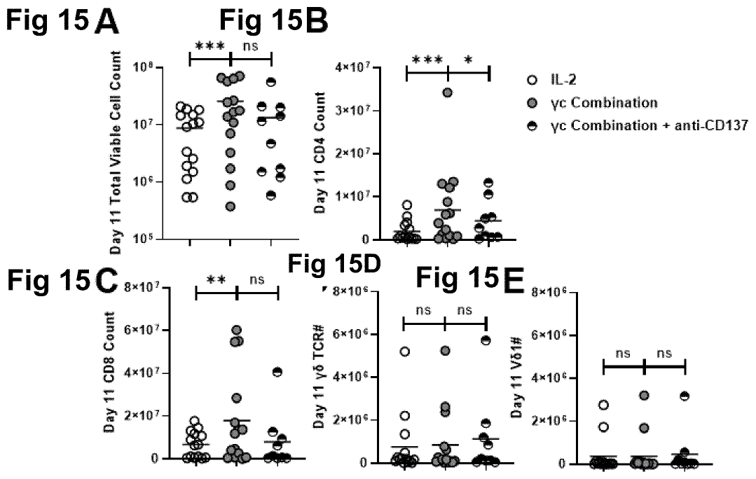

While the γ chain combination increased the total number of viable expanded TIL following 11 days of culture, due to increased CD3⁺αβ TCR⁺ CD4 and CD8 TIL, no differences in the number of γδ TCR⁺ or Vδ1⁺ cells were observed with or without CD137 stimulation compared to IL-2 alone (FIGS. 15A-E). Although no improvements in the expansion of γδ TIL were identified with use of the γ chain combination or CD137 engagement, these results support the continued utilization of high dose IL-2 or in combination with other γ chain cytokines during the pre-REP process to expand γδ TIL.

### Tumor Specific Vδ1 Infiltration and Survival Benefit

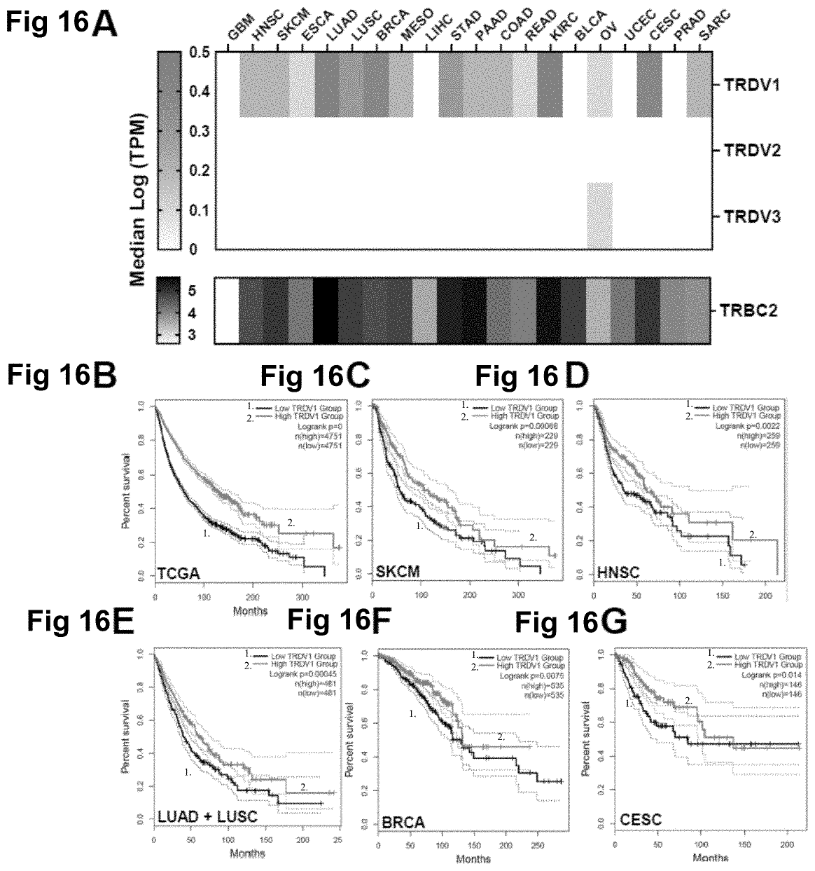

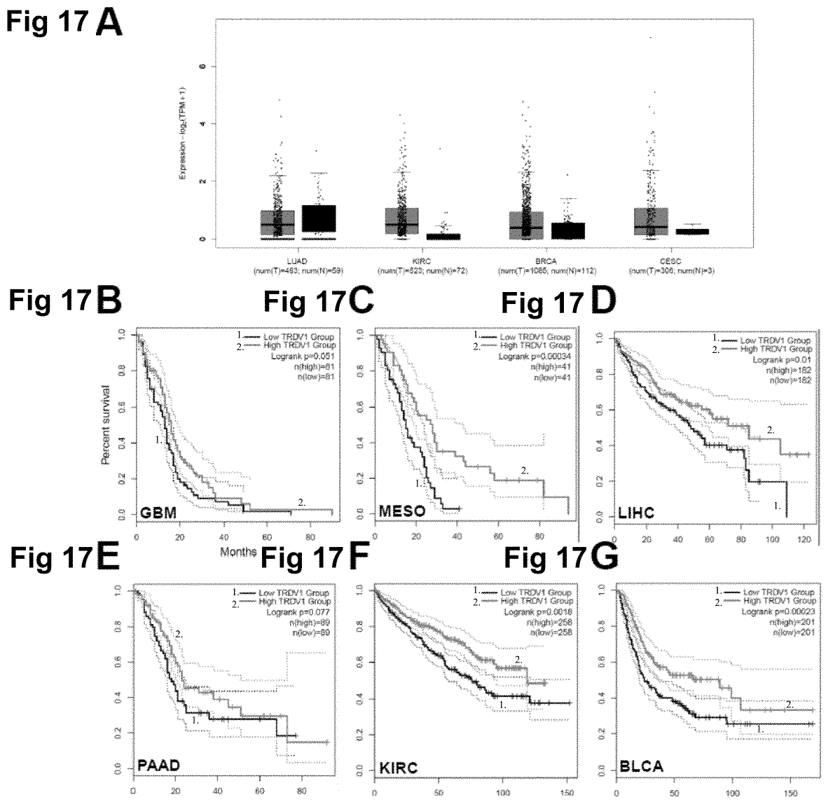

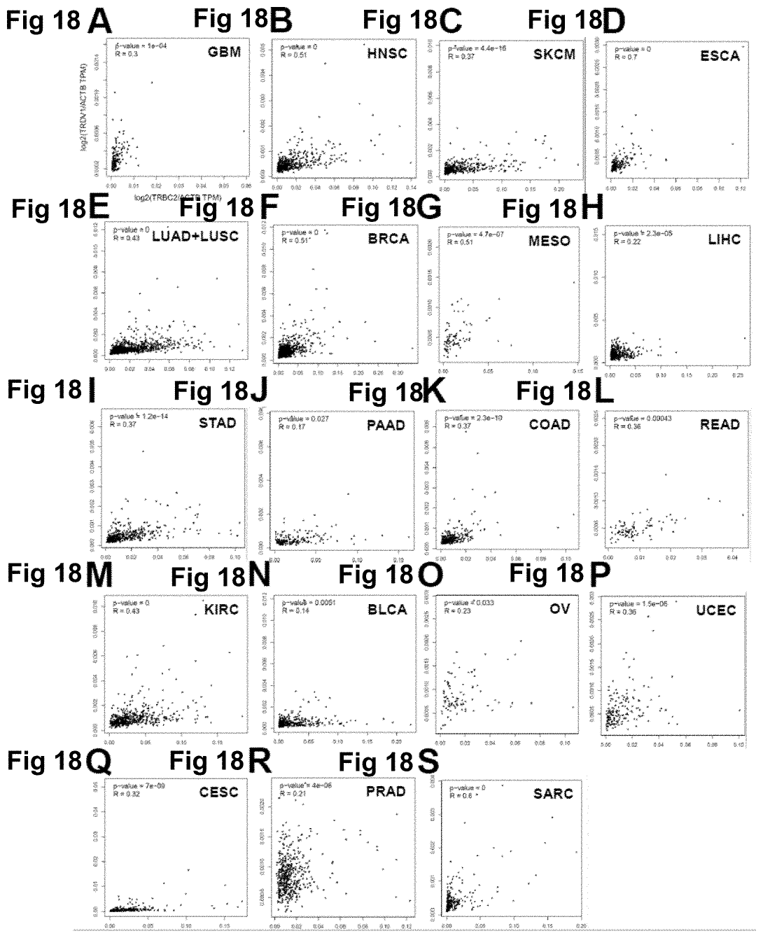

With multiple clinical studies of TIL therapy identifying infusion of increased number of tumor reactive T cells associated with objective clinical response, and the aforementioned sparse infiltration of γδ TIL, the following was performed to identify target indications with increased γδ TIL and determine their impact on long term survival (Radvanyi et al., Clin. Cancer Res., 18:6758-6770 (2012); Goff et al., J. Clin. Oncol., 34:2389-2397 (2016) and Chandran et al., Lancet Oncol., 18:792-802 (2017)). Using bulk RNA sequencing data of the 20 most prevalent solid tumors from The Cancer Genome Atlas (TCGA), the expression of γδ and αβ TIL was identified with the Gene Expression Profiling Interactive Analysis 2 (GEPIA 2) tool (FIG. 16A) (Tang et al., Nucleic Acids Res., 47:W556-W560 (2019); and Siegel et al., CA Cancer J Clin., 71:7-33 (2021)). The infiltration of the primary γδ T cell subsets (Vδ1⁺, Vδ2⁺, and Vδ3⁺ cells) were identified with the corresponding Vδ gene (TRDV1, TRDV2, and TRDV3), while αβ TIL were identified with the Vβ 2 constant region of the TCR (TRBC2). This method enabled clear identification of γδ TIL populations as previously utilized RNA gene signatures of γδ T cells have been shown to incorrectly include other immune effector subsets during classification of γδ T cells (Gentles et al., Nat. Med., 21:938-945 (2015); and Tosolini et al., Oncoimmunology, 6:e1284723 (2017)). Across most tumor types, the expression of TRDV1 trended to be higher than TRDV2 or TRDV3. Ovarian serous cystadenocarcinoma (OV) was the only indication to demonstrate notable expression of TRDV3. TRDV1 was differentially expressed across tumor types and was greatest in lung adenocarcinoma (LUAD, 0.5 median Log transcripts per million (TPM)), kidney renal cell carcinoma (KIRC, 0.5 Log TPM), breast carcinoma (BRCA, 0.4 Log TPM), and cervical squamous cell carcinoma and endocervical adenocarcinoma (CESC, 0.4 Log TPM). Full cohort TRDV1 expression for these selected tumors is shown in FIG. 17A. Glioblastoma (GBM), liver hepatocellular carcinoma (LIHC), bladder urothelial carcinoma (BLCA), uterine corpus endometrial carcinoma (UCEC), and prostate adenocarcinoma (PRAD) displayed the lowest expression of TRDV1, (median TRDV1 Log TPM=0). Across all tumor types, TRDV1 expression was positively correlated with expression of TRBC2 (FIGS. 18A-S).

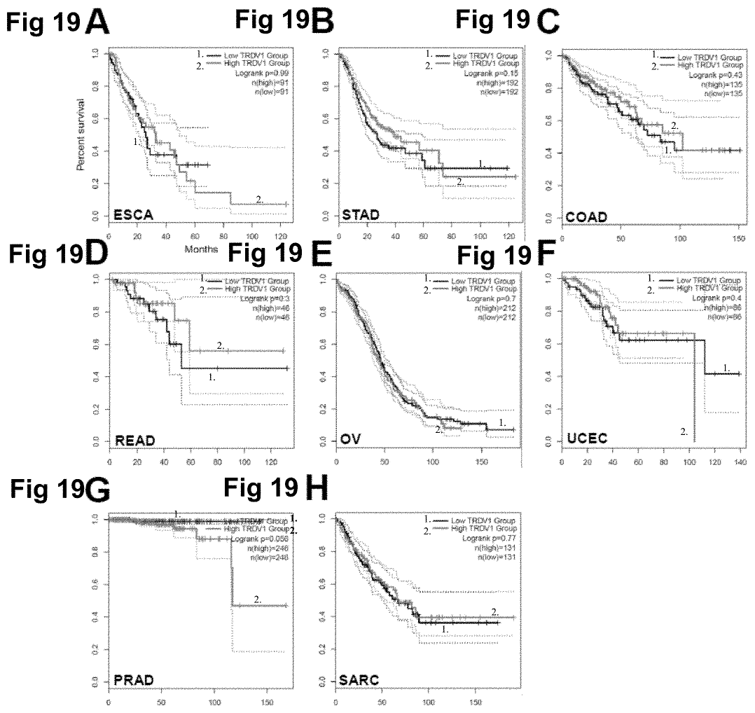

Given the predominant infiltration of the Vδ1 subset across tumors compared to the other γδ T cell subsets and association with autologous tumor reactivity, the prognostic impact of TRDV1 expression on overall survival in the selected tumors was evaluated. Following normalization of TRDV1 expression with beta actin (ACTB), the cohorts were split into high and low expression groups based on the median expression level of TRDV1 of individual tumor types. When including all TCGA tumors available for analysis on the GEPIA 2 server, high expression of TRDV1 was associated with considerable survival benefit (p<0.00001, FIG. 16B). High expression of TRDV1 was similarly associated with significant survival benefit in 12 of the 20 profiled solid tumors, including skin cutaneous melanoma (SKCM, p=0.0006), head and neck squamous cell carcinoma (HNSC, p=0.002), lung adenocarcinoma and lung squamous cell carcinoma (LUSC, p=0.0004), breast cancer BRCA (p=0.007), esophageal cancer CESC (p=0.014), and pancreatic ductal adenocarcinoma (PDAC, p=0.077), which are current indications utilizing TIL therapy (FIGS. 16C-G and 17B-G). Increased TRDV1 was also associated with survival benefit in patients with GBM, mesothelioma (MESO), LIHC, KIRC, and BLCA. In the remaining eight tumors, high expression of TRDV1 was not associated with substantive improvements in survival (FIGS. 19A-H).

Taken together, the results provided herein demonstrate that tumor infiltrating γδ T cells, displaying diverse, patient specific repertoires, tissue resident effector memory phenotypes, and superior autologous tumor reactivity, can be successfully expanded by themselves or in parallel with αβ TIL to unleash the full TCR repertoire against cancer.

### Example 2—Additional Embodiments

Embodiment 1. A method for producing a cell population comprising γδ T cells, wherein said method comprises culturing a first cell population comprising γδ T cells in the presence of IL-2, IL-4, and IL-15 for 8 to 21 days to obtain a second cell population, wherein said second cell population comprises at least 10 times more γδ T cells than said first cell population.
Embodiment 2. The method of Embodiment 1, wherein said γδ T cells are human cells.
Embodiment 3. The method of any one of Embodiments 1-2, wherein said γδ T cells are tumor infiltrating γδ T cells.
Embodiment 4. The method of any one of Embodiments 1-3, wherein said first cell population is:

(i) a population of tumor infiltrating γδ T cells obtained from (a) tissue comprising a tumor or (b) healthy tissue that was within 30 mm of a tumor,

(ii) a population of γδ T cells obtained from healthy tissue,

(iii) a population of γδ T cells obtained from infected tissue, or

(iv) a population of γδ T cells obtained from tissue harboring autoimmune T cells.

Embodiment 5. The method of Embodiment 4, wherein said method comprises obtaining said first cell population from said tissue comprising said tumor.
Embodiment 6. The method of Embodiment 4, wherein said method comprises obtaining said first cell population from said healthy tissue that was within 30 mm of said tumor.
Embodiment 7. The method of any one of Embodiments 1-6, wherein said first cell population is a cell population that was cultured in the presence of 50 international units/mL to 6000 international units/mL of IL-2 and in the absence of IL-4 and IL-15 for 3 to 15 days prior to said culturing in the presence of IL-2, IL-4, and IL-15.
Embodiment 8. The method of any one of Embodiments 1-6, wherein said first cell population is a cell population that was cultured in the presence of 100 international units/mL to 4000 international units/mL of IL-2 and in the absence of IL-4 and IL-15 for 8 to 15 days prior to said culturing in the presence of IL-2, IL-4, and IL-15.
Embodiment 9. The method of any one of Embodiments 1-8, wherein said first cell population is a cell population that was enriched for tumor infiltrating γδ T cells.
Embodiment 10. The method of any one of Embodiments 1-8, wherein said first cell population is a cell population that was enriched for tumor infiltrating γδ T cells via (a) the removal of at least some αβ T cells or (b) the isolation of at least some γδ T cells.
Embodiment 11. The method of any one of Embodiments 9-10, wherein said method comprises removing at least some αβ T cells from a cell population to obtain said first cell population.
Embodiment 12. The method of Embodiment 11, wherein said removing comprises positively selecting αβ T cells and removing the positively selected αβ T cells.
Embodiment 13. The method of any one of Embodiments 9-10, wherein said method comprises isolating at least some γδ T cells from a cell population to obtain said first cell population.
Embodiment 14. The method of Embodiment 13, wherein said isolating comprises positively selecting γδ T cells and isolating the positively selected γδ T cells.
Embodiment 15. The method of any one of Embodiments 1-14, wherein said culturing said first cell population comprising γδ T cells in the presence of IL-2, IL-4, and IL-15 for said 8 to 21 days comprises culturing said first cell population comprising γδ T cells in the presence of IL-2, IL-4, IL-15, irradiated PBMCs, and an anti-CD3 antibody for said 8 to 21 days.
Embodiment 16. The method of any one of Embodiments 1-15, wherein said culturing said first cell population comprising γδ T cells in the presence of IL-2, IL-4, and IL-15 is for 12 to 16 days.
Embodiment 17. The method of any one of Embodiments 1-15, wherein said culturing said first cell population comprising γδ T cells in the presence of IL-2, IL-4, and IL-15 is for 13 to 15 days.
Embodiment 18. The method of any one of Embodiments 1-17, wherein said second cell population comprises:

at least 50 times more γδ T cells than said first cell population,

at least 100 times more γδ T cells than said first cell population,

at least 200 times more γδ T cells than said first cell population,

at least 300 times more γδ T cells than said first cell population, or

at least 400 times more γδ T cells than said first cell population.

Embodiment 19. The method of any one of Embodiments 1-18, wherein said second cell population comprises greater than 1×10⁸ γδ T cells.
Embodiment 20. The method of any one of Embodiments 1-19, wherein said IL-2 is a human IL-2, wherein said IL-4 is a human IL-4, and wherein said IL-15 is a human IL-15.
Embodiment 21. The method of any one of Embodiments 1-20, wherein greater than 85 percent of the CD3⁺ cells of said second cell population are γδ TCR⁺ cells.
Embodiment 22. The method of any one of Embodiments 1-21, wherein less than 10 percent of the CD3⁺ cells of said second cell population are αβ TCR⁺ cells.
Embodiment 23. The method of any one of Embodiments 1-22, wherein less than 10 percent of the CD45⁺ cells of said second cell population are NK cells.
Embodiment 24. The method of any one of Embodiments 1-23, wherein greater than 30 percent of the γδ TCR⁺ cells of said second cell population are Vδ1⁺ cells.
Embodiment 25. The method of any one of Embodiments 1-24, wherein less than 60 percent of the γδ TCR⁺ cells of said second cell population are Vδ1⁻Vδ2⁻ cells.
Embodiment 26. The method of any one of Embodiments 1-25, wherein less than 25 percent of the γδ TCR⁺ cells of said second cell population are Vδ2⁺ cells.
Embodiment 27. The method of any one of Embodiments 1-26, wherein greater than 70 percent of the γδ TCR⁺ cells of said second cell population are TEM cells.
Embodiment 28. The method of any one of Embodiments 1-27, wherein less than 25 percent of the γδ TCR⁺ cells of said second cell population are T_{EMRA} cells.
Embodiment 29. The method of any one of Embodiments 1-28, wherein less than 10 percent of the γδ TCR⁺ cells of said second cell population are CD69⁺ CD103⁺ T_{RM} cells.
Embodiment 30. The method of any one of Embodiments 1-29, wherein from 1 to 10 percent of the γδ TCR⁺ cells of said second cell population are CD69⁺ CD103⁺ T_{RM} cells.
Embodiment 31. The method of any one of Embodiments 1-30, wherein less than 50 percent of the γδ TCR⁺ cells of said second cell population are CD56⁺ cells.
Embodiment 32. The method of any one of Embodiments 1-31, wherein from 1 to 50 percent of the γδ TCR⁺ cells of said second cell population are CD56⁺ cells.
Embodiment 33. The method of any one of Embodiments 1-32, wherein from 1 to 40 percent of the γδ TCR⁺ cells of said second cell population are CD137⁺ cells.
Embodiment 34. The method of any one of Embodiments 1-33, wherein less than 25 percent of the γδ TCR⁺ cells of said second cell population are PD-1⁺ cells.
Embodiment 35. The method of any one of Embodiments 1-34, wherein from 5 to 40 percent of the γδ TCR⁺ cells of said second cell population are BTLA⁺ cells.
Embodiment 36. The method of any one of Embodiments 1-35, wherein greater than 60 percent of the γδ TCR⁺ cells of said second cell population are NKG2D⁺ cells.
Embodiment 37. The method of any one of Embodiments 1-36, wherein greater than 20 percent of the γδ TCR⁺ cells of said second cell population are NKp46⁺ cells.
Embodiment 38. An isolated cell population comprising polyclonal γδ T cells, wherein said population comprises greater than 1×10⁸ γδ T cells.
Embodiment 39. The cell population of Embodiment 38, wherein greater than 85 percent of the CD3⁺ cells said cell population are γδ TCR⁺ cells.
Embodiment 40. The cell population of any one of Embodiments 38-39, wherein less than 10 percent of the CD3⁺ cells of said cell population are αβ TCR⁺ cells.
Embodiment 41. The cell population of any one of Embodiments 39-40, wherein less than 10 percent of the CD45⁺ cells of said cell population are NK cells.
Embodiment 42. The cell population of any one of Embodiments 39-41, wherein greater than 30 percent of the γδ TCR⁺ cells of said cell population are Vδ1⁺ cells.
Embodiment 43. The cell population of any one of Embodiments 39-42, wherein less than 60 percent of the γδ TCR⁺ cells of said cell population are Vδ1⁻Vδ2⁻ cells.
Embodiment 44. The cell population of any one of Embodiments 39-43, wherein less than 25 percent of the γδ TCR⁺ cells of said cell population are Vδ2⁺ cells.
Embodiment 45. The cell population of any one of Embodiments 39-44, wherein greater than 70 percent of the γδ TCR⁺ cells of said cell population are TEM cells.
Embodiment 46. The cell population of any one of Embodiments 39-45, wherein less than 25 percent of the γδ TCR⁺ cells of said cell population are T_{EMRA} cells.
Embodiment 47. The cell population of any one of Embodiments 39-46, wherein less than 10 percent of the γδ TCR⁺ cells of said cell population are CD69⁺ CD103⁺ T_{RM} cells.
Embodiment 48. The cell population of any one of Embodiments 39-47, wherein from 1 to 10 percent of the γδ TCR⁺ cells of said cell population are CD69⁺ CD103⁺ T_{RM} cells.
Embodiment 49. The cell population of any one of Embodiments 39-48, wherein less than 50 percent of the γδ TCR⁺ cells of said cell population are CD56⁺ cells.
Embodiment 50. The cell population of any one of Embodiments 39-49, wherein from 1 to 50 percent of the γδ TCR⁺ cells of said cell population are CD56⁺ cells.
Embodiment 51. The cell population of any one of Embodiments 39-50, wherein from 1 to 40 percent of the γδ TCR⁺ cells of said cell population are CD137⁺ cells.
Embodiment 52. The cell population of any one of Embodiments 39-51, wherein less than 25 percent of the γδ TCR⁺ cells of said cell population are PD-1⁺ cells.
Embodiment 53. The cell population of any one of Embodiments 39-52, wherein from 5 to 40 percent of the γδ TCR⁺ cells of said cell population are BTLA⁺ cells.
Embodiment 54. The cell population of any one of Embodiments 39-53, wherein greater than 60 percent of the γδ TCR⁺ cells of said cell population are NKG2D⁺ cells.
Embodiment 55. The cell population of any one of Embodiments 39-54, wherein greater than 20 percent of the γδ TCR⁺ cells of said cell population are NKp46⁺ cells.
Embodiment 56. The cell population of any one of Embodiments 39-55, wherein the cells of said cell population are human cells.
Embodiment 57. The cell population of any one of Embodiments 39-56, wherein said γδ T cells are tumor infiltrating γδ T cells.
Embodiment 58. The cell population of any one of Embodiments 39-57, wherein cell population was produced using the method of any one of Embodiments 1-37.
Embodiment 59. A method for providing a mammal with γδ T cells, wherein said method comprises administering a cell population produced as set forth in any one of Embodiments 1-37 to a mammal.
Embodiment 60. The method of Embodiment 59, wherein said mammal is a human.
Embodiment 61. The method of any one of Embodiments 59-60, wherein said mammal has cancer.
Embodiment 62. The method of any one of Embodiments 59-61, wherein the cells of said first cell population are allogenic or autologous to said mammal administered said cell population.
Embodiment 63. A method for providing a mammal with γδ T cells, wherein said method comprises administering said cell population of any one of Embodiments 38-58 to a mammal.
Embodiment 64. The method of Embodiment 63, wherein said mammal is a human.
Embodiment 65. The method of any one of Embodiments 63-64, wherein said mammal has cancer, an autoimmune condition, or an infection.
Embodiment 66. The method of any one of Embodiments 59-65, wherein the cells of said cell population are allogenic or autologous to said mammal.
Embodiment 67. A method for treating cancer, wherein said method comprises administering a cell population produced as set forth in any one of Embodiments 1-37 to a mammal having cancer.
Embodiment 68. The method of Embodiment 67, wherein said mammal is a human.
Embodiment 69. The method of any one of Embodiments 67-68, wherein the cells of said first cell population are allogenic or autologous to said mammal having cancer.
Embodiment 70. A method for treating cancer, wherein said method comprises administering said cell population of any one of Embodiments 38-58 to a mammal having cancer.
Embodiment 71. The method of Embodiment 70, wherein said mammal is a human.
Embodiment 72. The method of any one of Embodiments 70-71, wherein the cells of said cell population are allogenic or autologous to said mammal having cancer.
Embodiment 73. The method of any one of Embodiments 59-72, wherein said method comprises administering αβ T cells to said mammal.
Embodiment 74. A method for treating an autoimmune condition, wherein said method comprises administering said cell population of any one of Embodiments 38-58 to a mammal having an autoimmune condition.
Embodiment 75. The method of Embodiment 74, wherein said mammal is a human.
Embodiment 76. The method of any one of Embodiments 74-75, wherein the cells of said cell population are allogenic or autologous to said mammal having said autoimmune condition.
Embodiment 77. The method of any one of Embodiments 74-76, wherein said method comprises administering αβ T cells to said mammal.
Embodiment 78. A method for treating an infection, wherein said method comprises administering said cell population of any one of Embodiments 38-58 to a mammal having an infection.
Embodiment 79. The method of Embodiment 78, wherein said mammal is a human.
Embodiment 80. The method of any one of Embodiments 78-79, wherein the cells of said cell population are allogenic or autologous to said mammal having said infection.
Embodiment 81. The method of any one of Embodiments 78-80, wherein said method comprises administering αβ T cells to said mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for producing a cell population comprising γδ T cells, wherein said method comprises culturing a first cell population comprising γδ T cells in the presence of IL-2, IL-4, and IL-15 for 8 to 21 days to obtain a second cell population, wherein said second cell population comprises at least 10 times more γδ T cells than said first cell population.

2. The method of claim 1, wherein said γδ T cells are human cells.

3. The method of claim 1, wherein said method comprises obtaining said first cell population from said healthy tissue that was within 30 mm of said tumor.

4. The method of claim 1, wherein said first cell population is a cell population that was cultured in the presence of 50 international units/mL to 6000 international units/mL of IL-2 and in the absence of IL-4 and IL-15 for 3 to 15 days prior to said culturing in the presence of IL-2, IL-4, and IL-15.

5. The method of claim 1, wherein said first cell population is a cell population that was enriched for tumor infiltrating γδ T cells via (a) the removal of at least some αβ T cells or (b) the isolation of at least some γδ T cells.

6. The method of claim 5, wherein said method comprises removing at least some αβ T cells from a cell population to obtain said first cell population.

7. The method of claim 5, wherein said method comprises isolating at least some γδ T cells from a cell population to obtain said first cell population.

8. The method of claim 1, wherein said culturing said first cell population comprising γδ T cells in the presence of IL-2, IL-4, and IL-15 for said 8 to 21 days comprises culturing said first cell population comprising γδ T cells in the presence of IL-2, IL-4, IL-15, irradiated PBMCs, and an anti-CD3 antibody for said 8 to 21 days.

9. The method of claim 1, wherein said second cell population comprises at least 50 times more γδ T cells than said first cell population.

10. The method of claim 1, wherein greater than 85 percent of the CD3⁺ cells of said second cell population are γδ TCR⁺ cells.

11. An isolated cell population comprising polyclonal γδ T cells, wherein said population comprises greater than 1×10⁸ γδ T cells.

12. The cell population of claim 11, wherein greater than 85 percent of the CD3⁺ cells of said cell population are γδ TCR⁺ cells.

13. The cell population of claim 11, wherein less than 10 percent of the CD3⁺ cells of said cell population are αβ TCR⁺ cells.

14. The cell population of claim 11, wherein the cells of said cell population are human cells.

15. The cell population of claim 11, wherein cell population was produced using a method that comprises culturing a first cell population comprising γδ T cells in the presence of IL-2, IL-4, and IL-15 for 8 to 21 days to obtain a second cell population, wherein said second cell population comprises at least 10 times more γδ T cells than said first cell population.

16. A method for providing a mammal with γδ T cells, wherein said method comprises administering a cell population to said mammal, wherein said administered cell population was produced using a method that comprises culturing a first cell population comprising γδ T cells in the presence of IL-2, IL-4, and IL-15 for 8 to 21 days to obtain a second cell population, wherein said second cell population comprises at least 10 times more γδ T cells than said first cell population, and wherein said second cell population is said administered cell population.

17. The method of claim 16, wherein said mammal is a human.

18. A method for providing a mammal with γδ T cells, wherein said method comprises administering a cell population to said mammal, wherein said cell population comprises an isolated cell population comprising polyclonal γδ T cells, wherein said isolated cell population comprises greater than 1×10⁸ γδ T cells.

19. The method of claim 18, wherein said mammal is a human.

20. A method for treating cancer, wherein said method comprises administering a cell population to a mammal having cancer, wherein said administered cell population was produced using a method that comprises culturing a first cell population comprising γδ T cells in the presence of IL-2, IL-4, and IL-15 for 8 to 21 days to obtain a second cell population, wherein said second cell population comprises at least 10 times more γδ T cells than said first cell population, and wherein said second cell population is said administered cell population.

21. The method of claim 20, wherein said mammal is a human.

22. A method for treating cancer, wherein said method comprises administering a cell population to a mammal having cancer, wherein said cell population comprises an isolated cell population comprising polyclonal γδ T cells, wherein said isolated cell population comprises greater than 1×10⁸ γδ T cells.

23. The method of claim 22, wherein said mammal is a human.

24. A method for treating an autoimmune condition, wherein said method comprises administering a cell population to a mammal having an autoimmune condition, wherein said cell population comprises an isolated cell population comprising polyclonal γδ T cells, wherein said isolated cell population comprises greater than 1×10⁸ γδ T cells.

25. The method of claim 24, wherein said mammal is a human.

26. A method for treating an infection, wherein said method comprises administering a cell population to a mammal having an infection, wherein said cell population comprises an isolated cell population comprising polyclonal γδ T cells, wherein said isolated cell population comprises greater than 1×10⁸ γδ T cells.

27. The method of claim 27, wherein said mammal is a human.
